(19) United States
(12) Patent Application Publication
(10) Pub. No.: US 2025/0318767 A1
(43) Pub. Date: Oct. 16, 2025

(54) System and Method for Mental Diagnosis Using EEG

(71) Applicant: Georgia State University Research Foundation, Inc., Atlanta, GA (US)

(72) Inventors: Weizheng Yan, Atlanta, GA (US); Vince D. Calhoun, Decatur, GA (US); Jing Sui, Johns Creek, GA (US)

(21) Appl. No.: 18/870,080

(22) PCT Filed: Jun. 6, 2023
(86) PCT No.: PCT/US2023/067963

§ 371 Date: Nov. 27, 2024

Related U.S. Application Data
(60) Provisional application No. 63/349,296, filed on Jun. 6, 2022.

(51) Int. Cl. A61B 5/16; A61B 5/00; A61B 5/369; G06N 3/044; G16H 50/20; G16H 50/70

(52) U.S. Cl. CPC A61B 5/165; A61B 5/369; A61B 5/4076; A61B 5/7264; G06N 3/044; G16H 50/20; G16H 50/70

(57) ABSTRACT

A system for and a method of diagnosing a mental illness in a patient are disclosed. The method measures signals, such as EEG signals, on a patient and applies a trained machine learning model on these signals. The model classifies the patient as having a mental illness or of being a normal control. The results are communicated to a user. In addition, a sub-type of a mental disorder may be identified by using other machine learning techniques on the features of the signals, such as a neural network, clustering, dimension reduction, and visualization algorithms. One such technique is t-distributed stochastic neighbor embedding (t-SNE).

### CROSS-REFERENCE TO RELATED PATENT APPLICATION

The present patent application claims priority to U.S. Provisional Patent Application No. 63/349,296, filed Jun. 6, 2022, and entitled “System and Method for Mental Diagnosis using EEG”, the disclosure of which is incorporated herein by reference thereto.

### BACKGROUND OF THE INVENTION

Mental disorders are very costly not only for the affected individual and their family but also for the society as a whole. The World Health Organization estimates that over 1 billion people worldwide suffer from some form of mental disorder. Further, by the year 2030, over 6 trillion dollars may be spent on treating the mental disorders. A further 16 billion dollars may be lost through an estimated 12 billion days of work lost each year due to the burden of mental disorder.

In the Unites States of America alone, approximately one in five adults experiences a mental disorder in a given year, 18.1% of adults experience an anxiety disorder, such as posttraumatic stress disorder, obsessive-compulsive disorder and specific phobias, 6.9% of adults have at least one major depressive episode each year, and 1.1% of adults live with schizophrenia.

Major psychiatric disorders, including major depressive disorder (MDD), bipolar disorder (BP), and schizophrenia (SZ), are among most severe and impactful mental illnesses. They often have strong comorbidities and share substantial features. Approximately 85%-90% of patients with depression also experience symptoms of anxiety, and vice versa. The simultaneous presence of two or more psychiatric diseases are associated with greater severity, worse response to the pharmacological treatment and have a greater risk of suicide than either condition alone.

The consequences of the lack of treatment for these mental disorders are significant. In the United States of America, mental disorders are the third most common cause of hospitalization for both youth and adults aged 18-44. Suicide is the tenth leading cause of death, and the second leading cause of death for those aged 15-24. And each day, approximately 18-22 Armed Forces veterans die by suicide.

A key factor in treatment of mental disorders is proper diagnosis. Typically, the standard method of diagnosing mental disorders includes either the use of the Diagnostic and Statistical Manual of Mental Disorders, Fifth Edition, American Psychiatric Association, (2013), Arlington, Va. (“DSM” of “DSM-5”) or the International Statistical Classification of Diseases and Related Health Problems (ICD), Chapter 5: Mental and behavioral disorders, 10th Revision (ICD-10), 1994, Geneva: World Health Organization (“ICD”). Both of these standards primarily involve diagnosis using conversation with the patient regarding symptoms and behavior.

Therefore, various other diagnostic approaches employ identification of disease-related objective biomarkers or subtypes obtained using structural magnetic resonance imaging (sMRI), or functional connectivity features derived from resting-state functional MRI. For example, by analyzing canonical correlations between resting-state fMRI functional connectivity and Hamilton Depression Rating Scale (Ham-D or HAMD), biomarkers were extracted for MDD subtype identification and predicted transcranial magnetic stimulation (TMS) treatment outcomes.

At present, psychiatric diagnoses are based entirely on phenomenological descriptions, with no assay-based biological criteria underlying diagnosis. This has resulted in syndrome categories within serious mental illness that have overlapping symptom fields. The disadvantage of the diagnostic evaluations being only subjective—that is, based on the interviewer's own perceptions—may lower the diagnostic reliability and the resultant treatment and, sometimes, may result in two clinicians forming two different diagnoses of the same patient. For example, due to the overlapping clinical symptoms at onset, about 60% of BP patients are initially misdiagnosed as MDD and have to wait 5-10 years before receiving appropriate diagnoses.

In addition, the biological heterogeneity of psychiatric disorders has a substantial effect on treatment outcome as well, resulting in the unpredictability of therapeutic effects based on pretreatment clinical symptoms. For example, the treatment response of treatment-resistant major depressive disorder using transcranial magnetic stimulation (TMS) varies from 45% to 60%. Thus, obtaining the correct mental disorder diagnosis and then accurately predicting the TMS-treatment response is imperative to best support recovery of the patient. As for TMS treatment, currently, the treatment plan of the TMS is designed entirely based on pretreatment clinical symptoms, rather than objective biomarkers.

Therefore, an accurate diagnosis based on objective neuroimaging biomarkers, which could benefit from machine learning methods, is important for making an individualized treatment plan. However, the steps of acquiring these MRI neuroimages are complex, time consuming and expensive.

Accordingly, a need arises for systems and methods to easily and accurately predict a mental disorder and provide to the user a proper TMS-treatment response predictor.

### SUMMARY OF THE INVENTION

Aspects of the disclosure relate to systems and methods for diagnosing mental illness in a patient. An exemplary method for diagnosing mental illness in a patient may comprise the steps of measuring a plurality of encephalography signals from a patient. The method may further comprise feeding the plurality of encephalography signals into a trained multi-scale recurrent neural network which may classify the patient as having a selected mental illness of a plurality of mental illnesses. Then the method may communicate the selected mental illness to a user.

In an embodiment, the plurality of encephalography signals comprises at least one electroencephalography (EEG) signal or at least one magnetoencephalography (MEG) signal. The EEG signal may comprise at least one of the bilateral front (Fp1, F8) channels or the parietal-occipital (P7, P3, P8, PO4, O1, O2) channels. The plurality of mental illnesses may comprise major depressive disorder, bipolar disorder, schizophrenia, and a normal control. The method may further comprise extracting a feature from the multi-scale recurrent neural network and applying the feature to a second machine learning model to classify a sub-type of the selected mental illness. The method may further comprise incorporating other patient information as part of signals input into the trained multi-scale recurrent neural network.

In an embodiment, the method may further comprise, if the patient is diagnosed as having a major depressive disorder, reducing the dimensionality of the plurality of encephalography signals and visualizing the dimensionality-reduced plurality of encephalography signals. The method may further comprise classifying the patient as belonging to a sub-type of major depressive disorder, based on the visualization, and communicating the sub-type to the user. In an example, the sub-type may comprise a major depressive disorder treatable with transcranial magnetic stimulation. A method of dimensionality reduction and visualization may be t-distributed stochastic neighbor embedding (t-SNE). A sub-type may also be identified by the use of a clustering technique.

In an embodiment, this disclosure relates to a system for diagnosing a patient has having a mental illness. The system may comprise a computer with memory and a processor, a user device, and a measurement device. The system may measure, using the measurement device, a plurality of encephalography signals from a patient. The system may then feed, at the computer, the plurality of encephalography signals into a trained multi-scale recurrent neural network, which classifies the patient as having a selected mental illness of a plurality of mental illnesses. The system may then communicate the selected mental illness to the user device.

In an embodiment, the plurality of encephalography signals comprises at least one electroencephalography (EEG) signal or at least one magnetoencephalography (MEG) signal. The EEG signal may comprise at least one of the bilateral front (Fp1, F8) channels or the parietal-occipital (P7, P3, P8, PO4, O1, O2) channels. The plurality of mental illnesses may comprise major depressive disorder, bipolar disorder, schizophrenia, and a normal control. The computer may further perform the steps of extracting a feature from the multi-scale recurrent neural network and applying the feature to a second machine learning model to classify a sub-type of the selected mental illness. The computer may also perform the step of incorporating other patient information as part of signals input into the trained multi-scale recurrent neural network.

In an embodiment, if the patient is diagnosed as having a major depressive disorder, the computer may further perform the steps of reducing the dimensionality of the plurality of encephalography signals and visualizing the dimensionality-reduced plurality of encephalography signals. The computer may further perform the steps of classifying the patient as belonging to a sub-type of major depressive disorder, based on the visualization, and communicating the sub-type to the user. In an example, the sub-type may comprise a major depressive disorder treatable with transcranial magnetic stimulation. In an example, the dimensionality reduction and visualization may comprise t-distributed stochastic neighbor embedding (t-SNE). The sub-type may also be identified by the use of a clustering technique.

### BRIEF DESCRIPTION OF THE DRAWINGS

So that the manner in which the above recited features of the present invention can be understood in detail, a more particular description of the invention, briefly summarized above, may be had by reference to embodiments, some of which are illustrated in the appended drawings. It is to be noted, however, that the appended drawings illustrate only typical embodiments of this invention and the invention may admit to other equally effective embodiments.

FIG. 1 illustrates a flowchart of an exemplary method.

FIG. 2 illustrates an overview of an exemplary method.

FIG. 3 illustrates an exemplary classification including two MDD subtypes.

FIG. 4 illustrates an exemplary multi-scale recurrent neural network.

FIG. 5 illustrates a confusion matrix of 4 class classification.

FIG. 6 illustrates chance levels for classification accuracy.

FIG. 7 illustrates an exemplary t-SNE visualization.

FIG. 8 illustrates the top contributing EEG channels.

FIG. 9 illustrates an exemplary t-SNE visualization for two sub-types.

FIG. 10 illustrates an exemplary confusion matrix for TMS outcome prediction using rsEEG.

FIG. 11 illustrates an exemplary ROC curve of the TMS outcome prediction.

FIG. 12 illustrates an exemplary t-SNE visualization for HDRS total score reduction.

FIG. 13 illustrates an exemplary t-SNE visualization for HDRS-psychosis score before TMS.

FIG. 14 illustrates an exemplary t-SNE visualization for HDRS-anxiety score before TMS.

FIG. 15 illustrates a change in HDRS total score.

FIG. 16 illustrates a change in HDRS psychiatric depression.

FIG. 17 illustrates a change in HDRS loss of motivated behavior.

FIG. 18 illustrates a change in HDRS psychosis.

FIG. 19 illustrates a change in HDRS anxiety.

FIG. 20 illustrates a change in HDRS sleep disturbance.

FIG. 21 illustrates a confusion matrix for EEG probe locations.

FIG. 22 illustrates a confusion matrix for EEG probe locations.

FIG. 23 illustrates a confusion matrix for EEG probe locations.

FIG. 24 illustrates the diagonal of the frontal EEG probe locations.

FIG. 25 illustrates the diagonal of the parietal EEG probe locations.

FIG. 26 illustrates the diagonal of the temporal EEG probe locations.

FIG. 27 illustrates the diagonal of the occipital and insula EEG probe locations.

FIG. 28 illustrates an EEG probe amplitude for location Fp1.

FIG. 29 illustrates an EEG probe amplitude for location F8.

FIG. 30 illustrates an EEG probe amplitude for location P7.

FIG. 31 illustrates an EEG probe amplitude for location P3.

FIG. 32 illustrates an EEG probe amplitude for location P8.

FIG. 33 illustrates an EEG probe amplitude for location O1.

FIG. 34 illustrates an EEG probe amplitude for location O2.

FIG. 35 illustrates an EEG probe amplitude for location PO4.

FIG. 36 illustrates details of the flanker task.

FIG. 37 illustrates an exemplary computing device.

Other features of the present embodiments will be apparent from the Detailed Description that follows.

### DETAILED DESCRIPTION

In the following detailed description of the preferred embodiments, reference is made to the accompanying drawings, which form a part hereof, and within which are shown by way of illustration specific embodiments by which the invention may be practiced. It is to be understood that other embodiments may be utilized and structural changes may be made without departing from the scope of the invention. Electrical, mechanical, logical, and structural changes may be made to the embodiments without departing from the spirit and scope of the present teachings. The following detailed description is therefore not to be taken in a limiting sense, and the scope of the present disclosure is defined by the appended claims and their equivalents.

The present disclosure relates to diagnosing major psychiatric disorders including major depressive disorder (MDD), bipolar disorder (BP), and schizophrenia (SZ) as distinct from normal controls (NC). These disorders are among the most severe and impactful mental illnesses. They lead to decreased quality of life, premature death and disability in many patients, and increased health care costs. Major psychiatric disorders often have strong comorbidities and shared substantial features, causing difficulties in diagnosis and treatment. The current psychiatric diagnosis guidelines are based on phenomenological descriptions, with no assay-based biological criteria underlying diagnosis. This has resulted in syndrome categories within major psychiatric disorders that have overlapping symptom fields. For instance, due to the overlapping clinical symptoms at onset, about 60% of BP patients are initially misdiagnosed as MDD and have to wait 5-10 years before receiving appropriate diagnoses. The biological heterogeneity of psychiatric disorders has a substantial effect on treatment outcomes, resulting in the unpredictability of therapeutic effects. For example, the treatment response of treatment-resistant major depressive disorder using transcranial magnetic stimulation (TMS) varies from 45% to 60%. By defining clinical-relevant “disease subtypes” based on biological biomarkers, more accurate therapy plans are promised to be made according to the characteristics of each subtype. Therefore, studying the heterogeneity and subtypes of psychiatric disorders based on objective biomarkers is urgent for accurate diagnosis and treatment.

Recent studies have adopted various approaches that transcend traditional diagnostic boundaries for identifying disease-related biomarkers or subtypes by using structural magnetic resonance imaging (sMRI), functional connectivity features derived from resting-state functional MRI, or resting-state electroencephalography (rsEEG). For example, two clinically relevant subtypes of post-traumatic stress disorder and MDD were identified based on functional connectivity patterns in EEG. By analyzing canonical correlations between resting-state fMRI functional connectivity and the Hamilton Depression Rating Scale, researchers extracted biomarkers and identified MDD subtypes of which TMS treatment outcomes could be predicted. (Drysdale, A. T. et aL. “Resting-state connectivity biomarkers define neurophysiological subtypes of depression.” Nat Med 23, 28-38, (2017).) Compared to MRI, EEG is an inexpensive measurement with higher time resolution and non-magnetic effects, making it a practical healthcare tool in a variety of clinical environments. Decades of studies on EEG have provided us with multiple methods (e.g., coherence, phase synchronization, phase-slope index, Granger causality) to quantify neural interactions as well as to provide a valid interpretation of the findings. However, due to the respective assumptions, the EEG metrics have limitations in fully leveraging the spatiotemporal information in EEG. Besides, the choice of a variety of hyperparameters such as frequency band makes the metrics challenging for subtype discovery. Moreover, non-linear approaches which are promising in extracting discriminative information from the time domain of rsEEG have not been well studied in the literature. Compared to the standard machine learning method, deep learning can encode more robust discriminative neuroimaging representations by characterizing potentially non-linear high-level patterns existing in the input features. The convolutional recurrent neural network, a specific deep learning architecture, has been proved efficient in mental disorder classification tasks by leveraging spatiotemporal information from fMRI time sequences. Different from the popular frequency-based EEG methods as reported by others, which separated EEG into predefined frequency bands, the convolutional recurrent neural network can automatically learn convolutional filters to extract the weighted combinations of EEG channels and then use the recurrent module to process sequential information for accurate classification and subtype discovery.

This disclosure describes a state-of-the-art deep learning method which employs a multi-scale convolutional recurrent neural network, MCRNN, to leverage the spatiotemporal information of rsEEG for multi-disease classification and subtype discovery. By initially learning profiles from three major psychiatric disorders with comorbidity comprehensively, the model can effectively inhibit the confounds (e.g. age, gender, scanners) by mapping the EEG features into the psychiatric-specific subspace, making unsupervised clustering practical for subtype discovery. In the example presented below, the model achieved and appreciable average 4-class classification accuracy (52.4%) when applied to a multiple psychiatric disorder dataset consisting of BP, MDD, SZ, and normal controls (NC). The knowledge learned by the deep learning model may be further used for clustering MDD patients. By applying the model to an independent validation dataset that consists of MDD, the MDD subtype which is sensitive to the TMS treatment was discovered, showing great promise in promoting the understanding of major psychiatric disorders and facilitating the individualized treatment of depression.

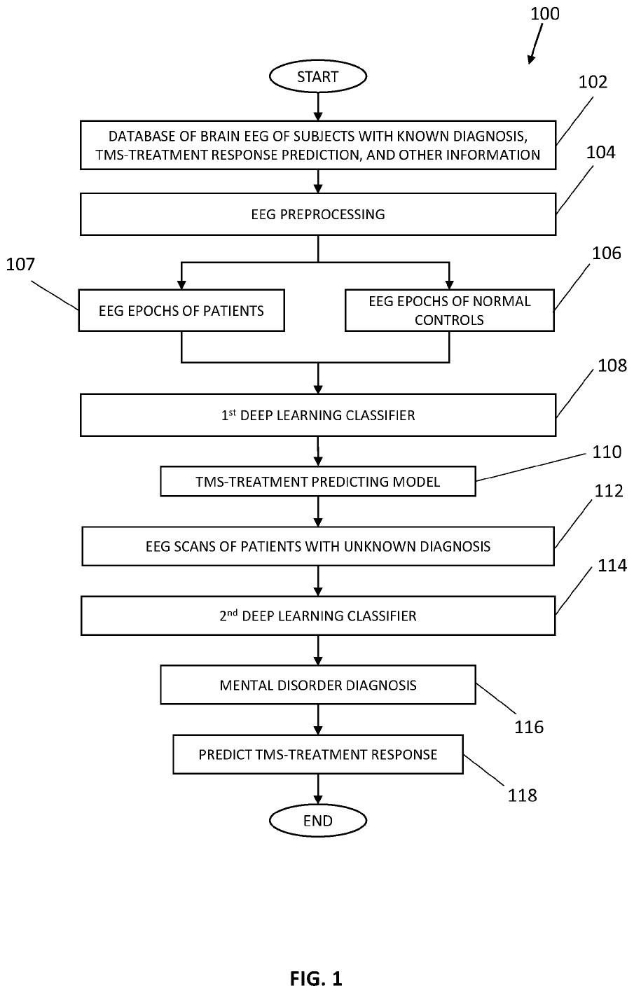

An overview of the method 100 is outlined in FIG. 1. A database of EEG measurements on human subjects with known diagnoses was generated and later accessed at step 102. In addition, also at step 102, other known information about the patients may be included, such as the patients' sex and age and also their response to TMS-treatment of MDD. The EEG data is accessed and pre-processed at step 104. The EEG data is divided into training and testing groups, and then used to train a deep learning classifier at step 108. In addition, at step 106, EEG measurements from human patients classed as normal (NC) may also be used in addition to the data from subjects with a known mental illness diagnosis 107. Both sets of data (normal controls and patients diagnosed with a known mental illness) may be used to train the deep learning classifier at step 108. Once the classifier has been trained and tested, it may be used to help develop an additional model to predict the TMS-treatment outcome at step 110. This additional model may use EEG scans of patients with an unknown diagnosis at step 112. These scans are pre-processed also at step 112 and then fed into the original trained model at step 114. A diagnosis or classification for a mental disorder class (e.g., MDD, BP, SZ, or NC) for each patient's EEG scans may be made at step 116. After these EEG scans have been classified, a prediction as to the TMS-treatment response may be made for patients classified in the MDD group at step 118.

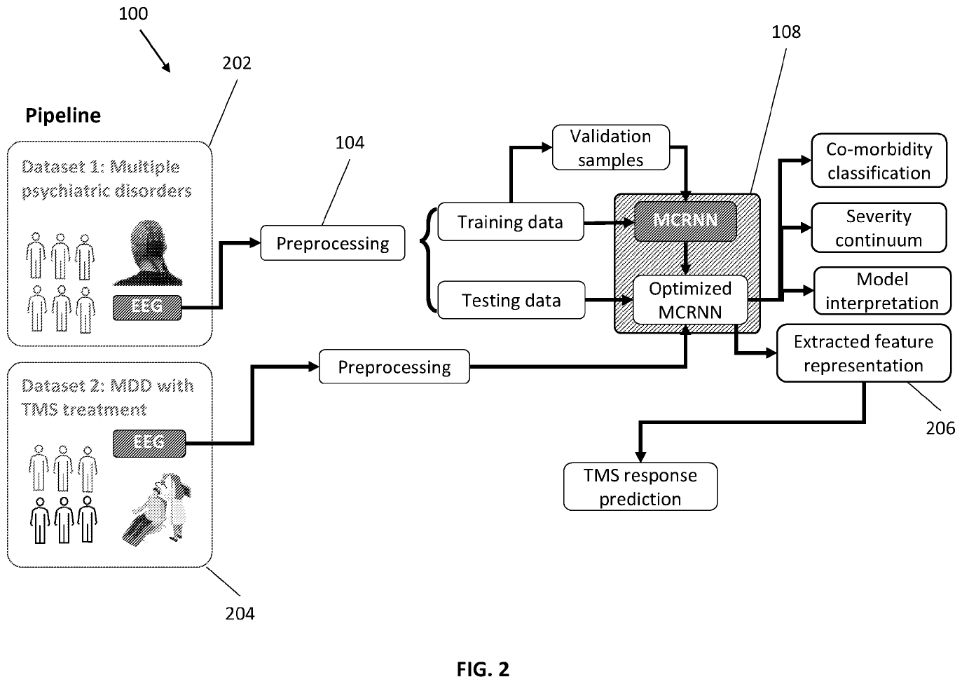

As shown in FIG. 2, two datasets may be used: a first dataset 202 of rsEEG samplings from patients with multiple psychiatric disorders and a second dataset 204 of samples of patients with major depressive disorder who were treated with TMS. In an example, the first dataset of 4-class rsEEG samplings were preprocessed 104 and then sent to a multi-scale recurrent neural network (MCRNN) for model optimization 108. After the preprocessing, EEG samples in the first dataset 202 were used for optimizing the parameters of MCRNN. A leave-one-out strategy may be used for evaluating the model performance. The severity continuum of multiple psychiatric disorders was visualized. The model interpretation was applied for discovering the most discriminative EEG channels. The second dataset 204 was sent to the optimized MCRNN for feature extraction, and the extracted features 206 were further used for unsupervised MDD subtype discovery. Two clustered subtypes are compared in the aspects of TMS response prediction 118, HDRS 120, functional connectivity 122, and neural plasticity 124.

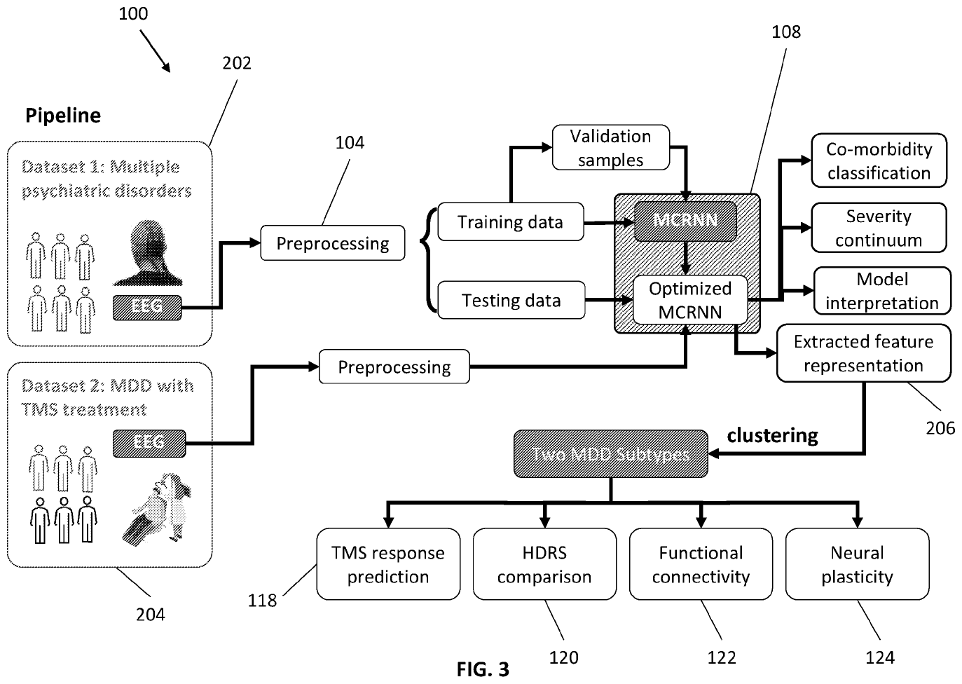

The leave-one-out cross-validation strategy was used for evaluating the classification performance. The severity continuum of psychiatric disorders was visualized based on the high-level feature learning by MCRNN. To identify the most discriminative EEG channels, an occlusion strategy was applied for explaining the discriminative power of EEG channels. To identify the TMS-response subtype, the optimized MCRNN model was further applied to an independent validation MDD dataset (the second dataset 204: for example, 25 subjects with TMS treatment), and two MDD subtypes were discovered by unsupervised clustering 118 of the features extracted using MCRNN, as shown in FIG. 3. The two MDD subtypes were compared in TMS treatment response 118, Hamilton Depression Rating Scale (HDRS) 120, functional connectivity 122, and neural plasticity 124.

### Multi-Scale Convolutional Recurrent Neural Network (MCRNN)

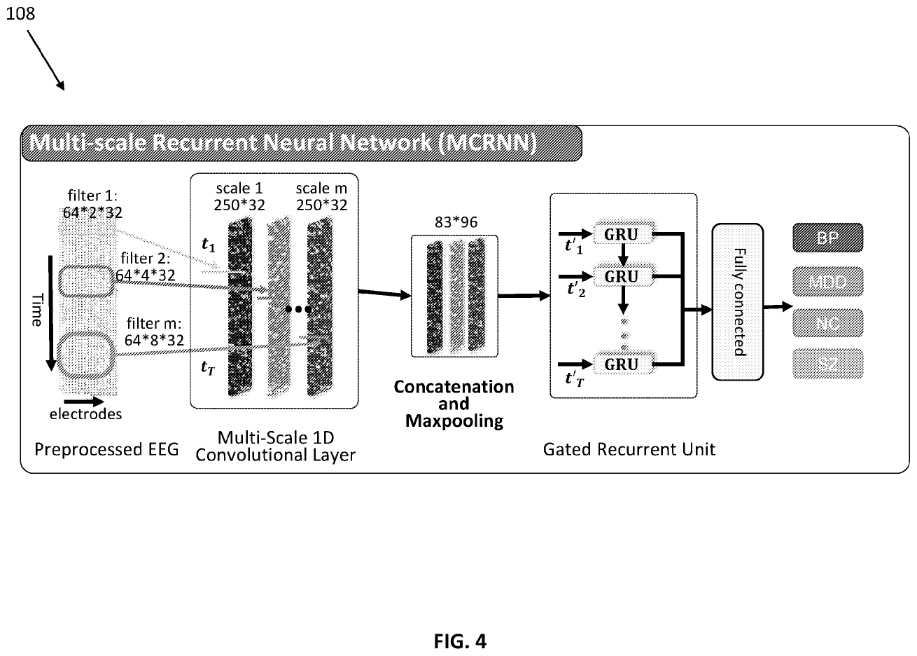

Some details of the MCRNN 108 are shown in FIG. 4. EEG voltage signals were measured as a function of time. Various filters were applied to these signals in a set of multi-scale convolutions. The model has two main modules: multi-scale 1D convolutional layers as filters to map the preprocessed EEG sampling into various feature spaces and a gated recurrent unit (GRU) for aggregating sequential information. The fully connected layer at the end will classify the input set of EEG voltage signals as belonging to one of the four categories: BP, MDD, NC, or SZ.

In the example shown in FIG. 4, the MCRNN 108 may consist of multiple 1D convolutional (Conv1D) filters with different scales, one concatenation layer, one max-pooling layer, a gated recurrent unit (GRU), and an averaged layer for integrating the spatiotemporal information for classification. The preprocessed EEG signals were fed into the proposed MCRNN model for parameter optimization. After optimizing the parameters, the model was saved for performance evaluation. The detailed architecture and mechanisms of the MCRNN model are as follows: The multi-scale 1D convolutional layer expands upon simple convolutional layers by including multiple filters of varying sizes in each Conv1D layer. The filter lengths used in the Conv1D are drawn from a logarithmic instead of a linear scale, leading to exponentially varying filter lengths (2, 4, and 8). Therefore, the dimensions of 3 different scales of convolutional filters are 64 (EEG channels)×2 (filter length)×32 (number of filters), 64×4×32, and 64×8×32. A concatenation layer then concatenates the incoming features among the depth axis, resulting in feature maps whose sizes are 250 (time points)×96 (feature dimensions). Whereafter, a max-pooling layer performs a down-sampling operation along the time dimensions with filter size 3, resulting in features whose size is 83 (time points)×96 (feature dimensions). The downsampled features are the input of the subsequent GRU layers. As for the GRU layer, the size of the GRU's hidden state was set to 32. The GRU layer can extract the sequential information and hidden states of the EEG signals. The extracted hidden states are then sent to the average-pooling layer to combine all GRU steps. The fully connected layer and SoftMax are then applied to get the final prediction results. More details of the model implementation are presented below.

### Details of the MCRNN Model Implementation

The MCRNN model was trained by minimizing the cross-entropy loss using the Adam optimizer. The training batch size was set to 512. The learning rate started from 0.001 and decayed after each epoch with a decay rate of 10⁻². To improve a generalization of the performance of the model and to overcome overfitting, dropout (dropout rate: 0.5 in the convolutional module, 0.3 in the GRU module, 0.5 in the fully connected layer) and L_{1,2-norm} regularization (GRU kernel regular l₁=10⁻⁴, l₂=10⁻⁴) were also applied for regulating the model parameters. The training process was stopped when the validation loss stopped decreasing for 50 epochs or when the maximum epochs (1000 epochs) had been executed. The intermediate model which achieved the highest accuracy on the validation dataset was reserved for testing. The proposed models were implemented on TensorFlow platform.

Each epoch is represented with a T×D matrix, where T is the length of samplings and D is the amount of EEG channels. To quantify the classification contribution of the dₜₕ channel, the samplings of dₜₕ channel were replaced with its averaged value while keeping other channels' samplings as they were. This replacement is equivalent to eliminating the contribution of dₜₕ channel. All the testing samples were processed in the same way and subsequently fed to the trained MCRNN model. The classification performance of the trained model which is fed with reduced features would decrease compared to the performance using all features. The channels which maximize the decrease of the classification performance are further selected as the most discriminative channels.

### Four-Class Classification Results and Psychiatric Spectrum Visualization

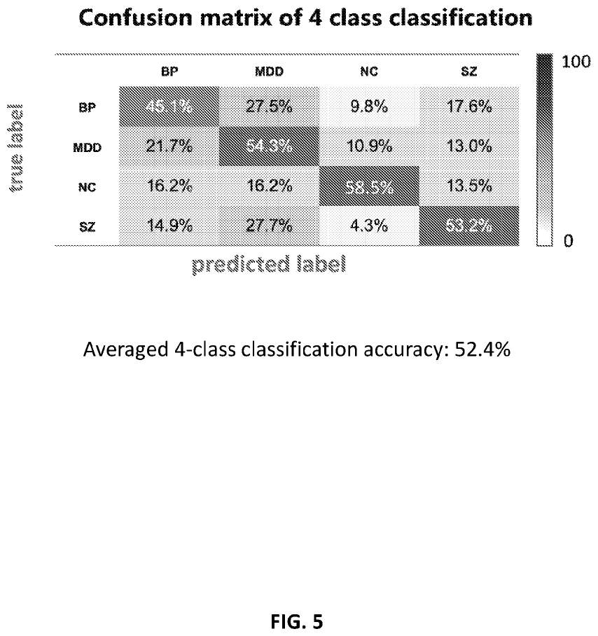

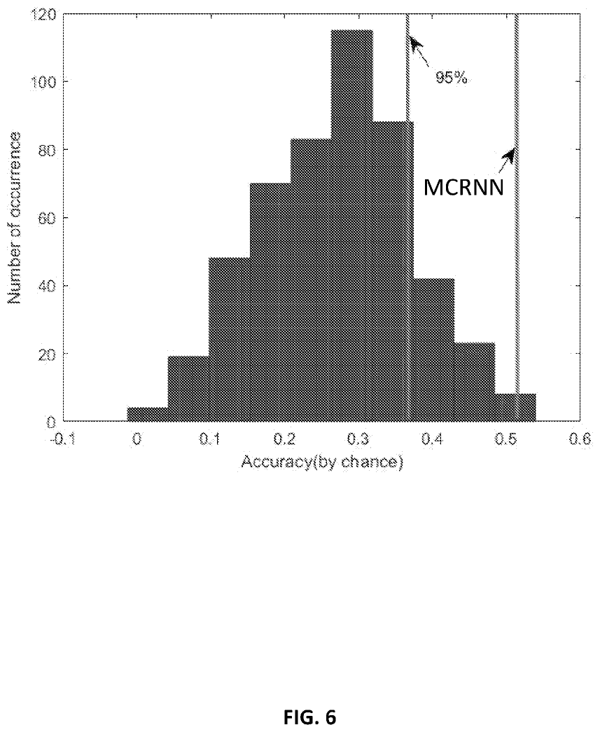

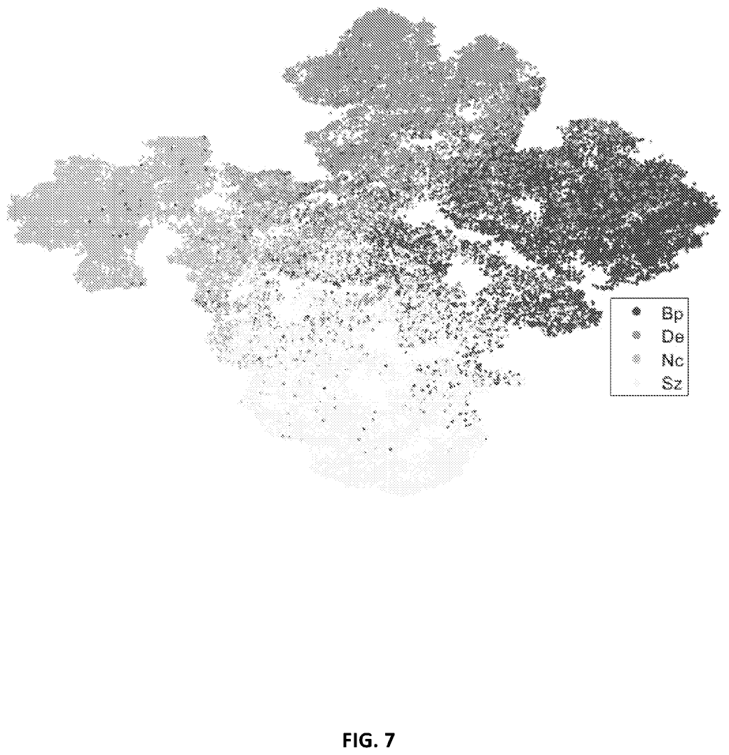

In an example, the first dataset 202 consisted of 185 subjects (51 BP, 46 MDD, 41 SZ, 47 NC=normal control) was used for training the MCRNN model. The leave-one-out strategy was applied for evaluating the classification performance. The average accuracy of the 4-class classification achieved 52.4% (FIG. 5). Table 1 shows the number of patients and corresponds to FIG. 5. The confusion matrix shows that MDD and BP exhibit more overlap than other psychiatric disorders. Permutation tests of overall accuracy for the classifier were conducted and the results shown in FIG. 6. For each permutation run, the labels of subjects were randomly shuffled. The result shows the null distributions of four-class classification accuracy from the empirical tests peaks at 0.28±0.06 (95% confidence intervals), whereas the MCRNN is at approximately 0.51, indicating that it is highly improbable that the MCRNN results are the result of chance level. To visualize the severity continuum of various mental disorders, a t-distributed stochastic neighbor embedding (t-SNE) was applied to the 32-dimensional features extracted from the second-last layer of MCRNN. FIG. 7 shows the “spectrum” of multiple psychiatric disorders. The severity spectrum shows that MDD and BP overlap, coinciding with the confusion matrix results.

TABLE 1
Confusion matrix of four class classification,
absolute number of patients

Absolute number

| | | BP | MDD | NC | SZ |
|---|---|---|---|---|---|
| True | BP | 23 | 14 | 5 | 9 |
| label | MDD | 10 | 25 | 5 | 6 |
| | NC | 6 | 6 | 24 | 5 |
| | SZ | 7 | 13 | 2 | 25 |

Predicted label

### Discriminative Features Discovery

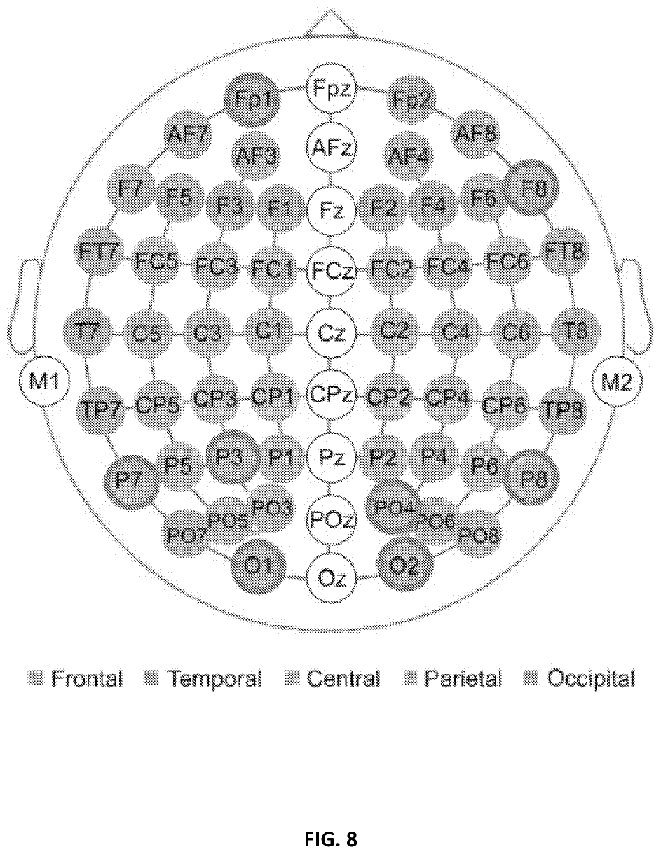

All epochs of 185 subjects in the first dataset 202 were used for optimizing the parameters of MCRNN. After optimizing the trained model, the parameters of the trained model were saved. All EEG epochs without removing any channel were sent to the MCRNN for obtaining the benchmark classification performance. Afterward, the EEG epochs with removed information of one specific channel were fed to the model for obtaining the decline of classification accuracy repeatedly. Then, the decline of accuracy when removing a specific channel was recorded and sorted. In this way, the contribution of each channel was calculated. As shown in FIG. 8, the most discriminative channels are located in Occipital (O1, O2, PO4), Parietal (P3, P7, P8), and Frontal (Fp1, F8). These most discriminative channels are highlighted with thicker borders in the figure. Details of the EEG electrodes can be found elsewhere in this disclosure.

### MDD Subtype Discovery

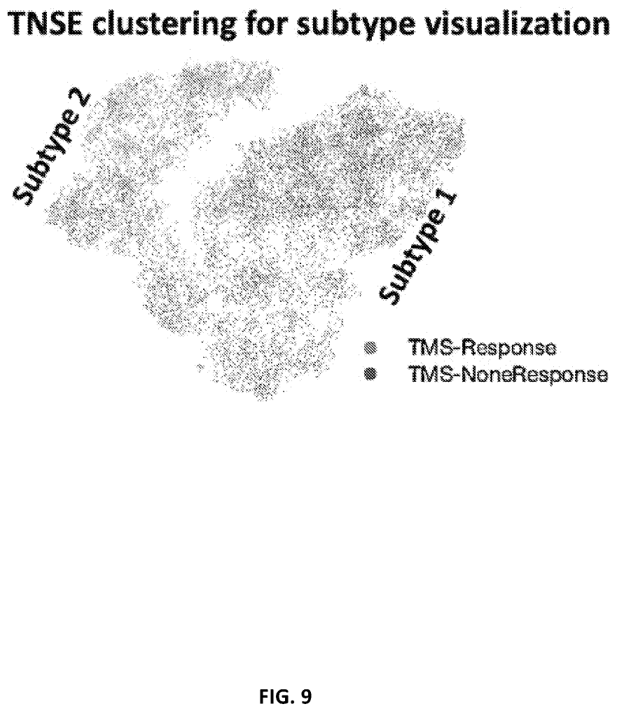

The understanding of psychiatric disorders with neurobiological heterogeneity could benefit from the identification of subtypes discovered using state-of-the-art neuroimaging techniques. Deep learning, which can learn a comprehensible task-specific projection spectrum from the high-dimensional samples, is promising in subtype discovery. In this example, after training the MCRNN model on the first dataset 202, the optimized MCRNN learned the mapping to project the EEG epochs to a subspace in which the psychiatric disorders are maximally separated. Afterwards, when the EEG epochs of the independent validation dataset (the second dataset 204) were sent to the optimized MCRNN, the epochs were mapped to the psychiatric-related subspace in which the non-psychiatric confounds (e.g., age, gender, scanners) are maximally inhibited, making the extracted features from MCRNN robust for subsequent subtype discovery. The t-SNE dimension reduction and visualization algorithm was subsequently applied to the extracted features, though other clustering algorithms may also be used. Two distinct groups are shown after this method was applied (FIG. 9). The two subtypes are further compared in the following four aspects: (1) TMS treatment response; (2) Hamilton depression rating scale; (3) functional connectivity patterns; (4) neural plasticity.

### TMS Treatment Response

It is very challenging to predict the TMS response with the original EEG signals because of their low signal-to-noise ratios. Therefore, the high-level features were extracted first from the trained MCRNN model and then these features were used to train a support vector machine (SVM) model to predict the TMS-treatment response.

During the training procedure, the MCRNN was firstly optimized using the training dataset. After training, the parameters of the model were saved (“MCRNN_trained”). The high-level features of the training dataset were extracted using the trained model. An SVM was then used to predict the TMS-response using these high-level features. A leave-one-out cross-validation (LOOCV) strategy was employed to evaluate the SVM prediction performance. Since each subject has 260˜300 epochs, during the training procedure, the label of the epoch was set to the same label as the subject when training the classifiers. The predicted results of epochs were used to vote and obtain the final label of the subjects when validating. The hyperparameters are also saved (“SVM_trained”) to the model.

When the model was applied to a new undiagnosed patient for diagnosis (classification) and TMS-treatment response prediction, the preprocessed EEG epochs are firstly sent to the “MCRNN_trained” model for high-level feature extraction. Afterwards, these extracted high-level features are then sent to the “SVM_trained” model to get the TMS-treatment response prediction.

TABLE 2
Confusion matrix for TMS outcome
using rsEEG, number of patients

True Label

| | | Non-Response | Response |
|---|---|---|---|
| Predicted | Non-Response | 16 | 1 |
| Label | Response | 3 | 5 |

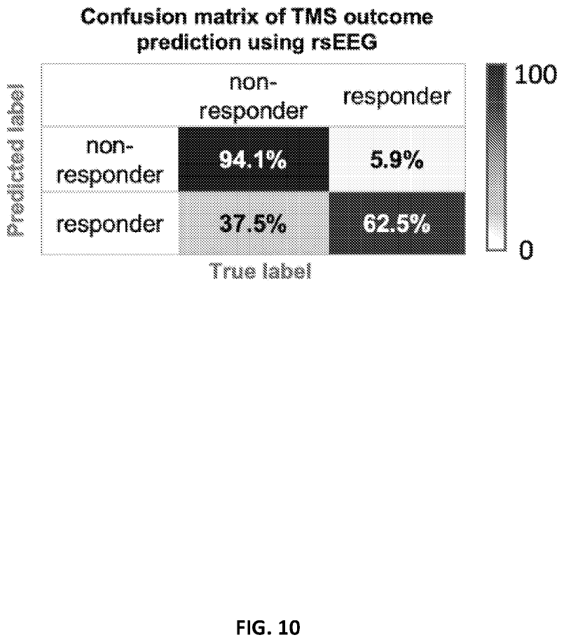

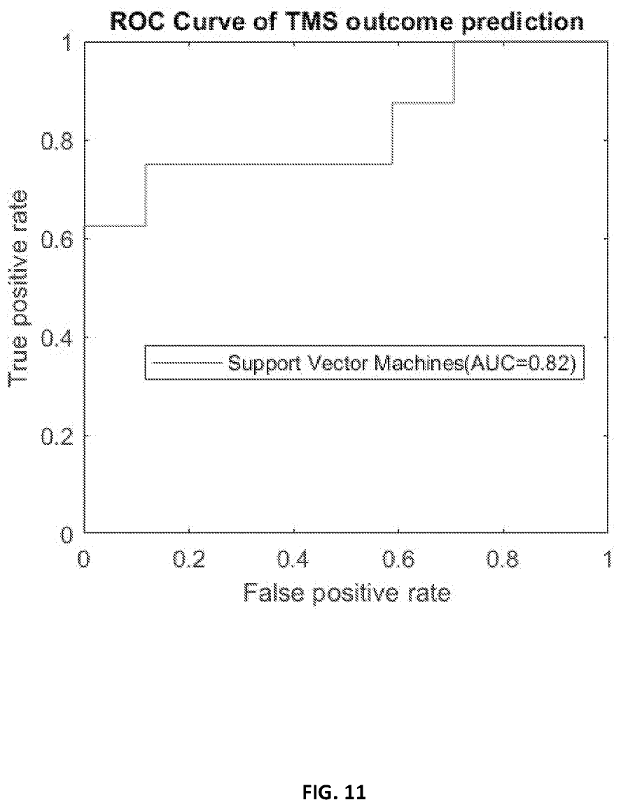

As shown in FIG. 10 and Table 2, the clustered subtypes are highly correlated to the TMS treatment response, indicating that the TMS response may be predicted before treatment using rsEEG. To further quantify the ability of extracted rsEEG features in predicting the TMS treatment response, a support vector machine (SVM) and leave-one-out cross-validation (LOOCV) strategy was applied to evaluate the prediction performance using MATLAB. The kernel function of the SVM was “radial basis function” and other SVM parameters were set as default. Since each subject had 260˜300 epochs, during the SVM training procedure, the label of each epoch was set the same as its corresponding subject. During testing, the predicted results of epochs were used to vote for obtaining the final label of the subject. The classification result is shown in FIG. 10 and Table 2 and the receiver operating characteristic (ROC) is shown in FIG. 11. The TMS treatment response could be predicted using SVM based on high-level rsEEG features extracted using MCRNN with a mean accuracy of 84%. The area under the curve (AUC) is 0.82, which also demonstrates that this method is considered highly accurate.

### Depressive Symptom Comparison

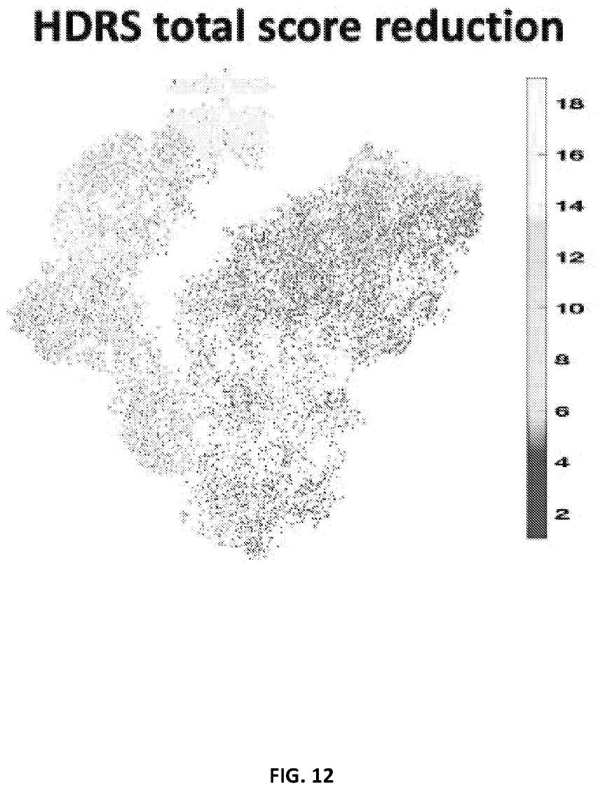

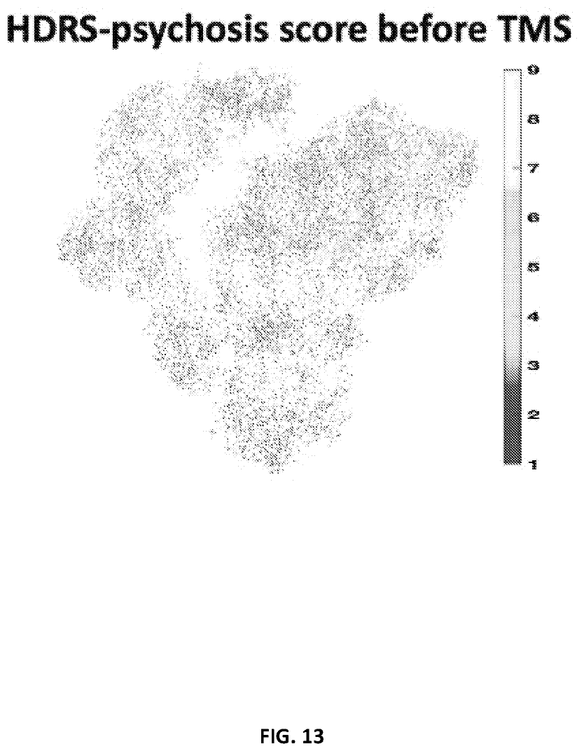

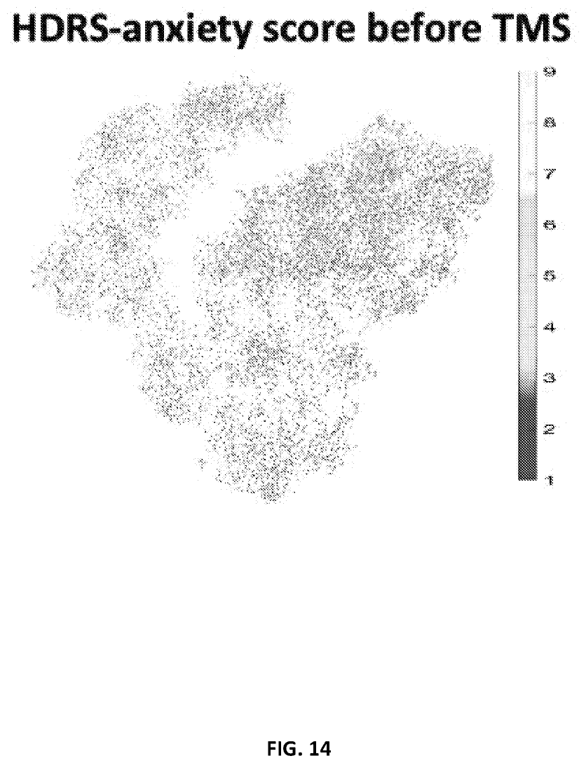

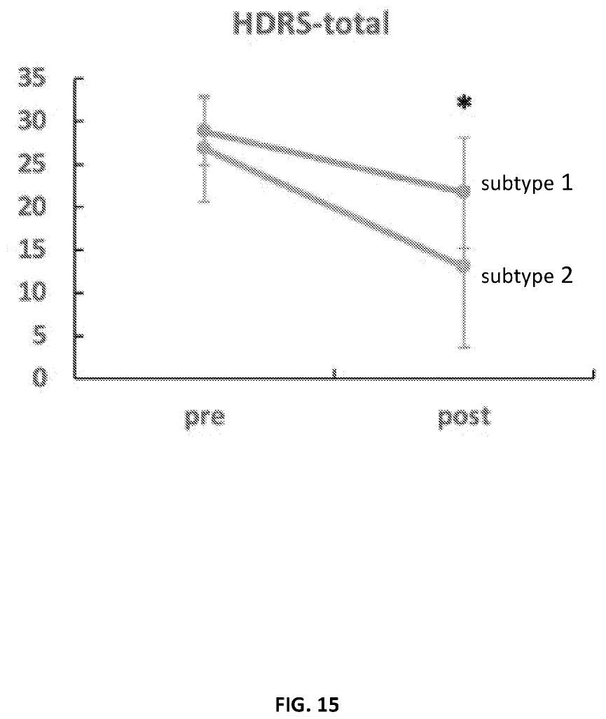

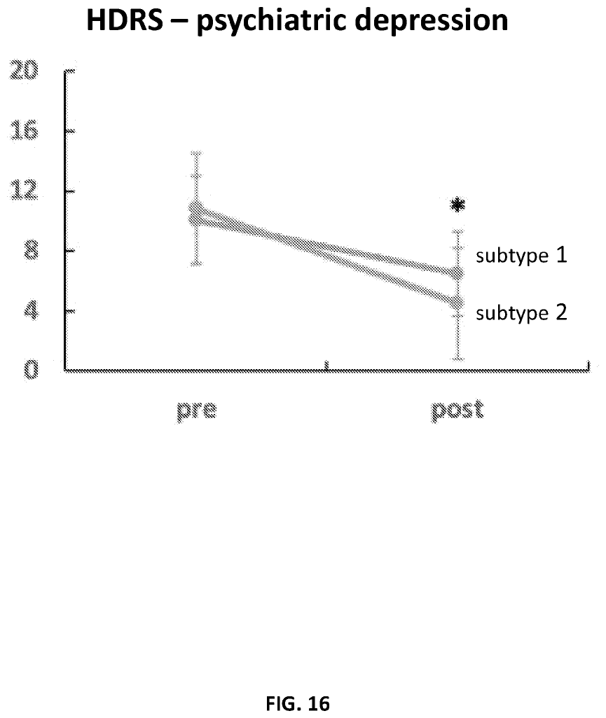

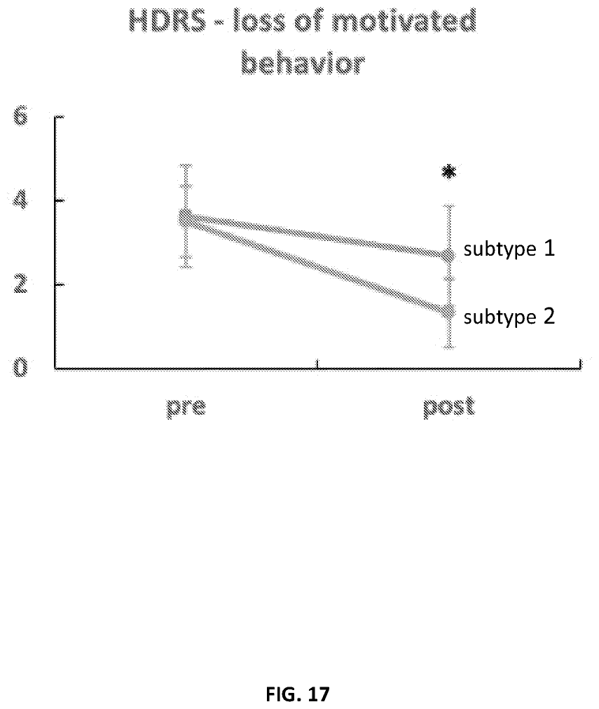

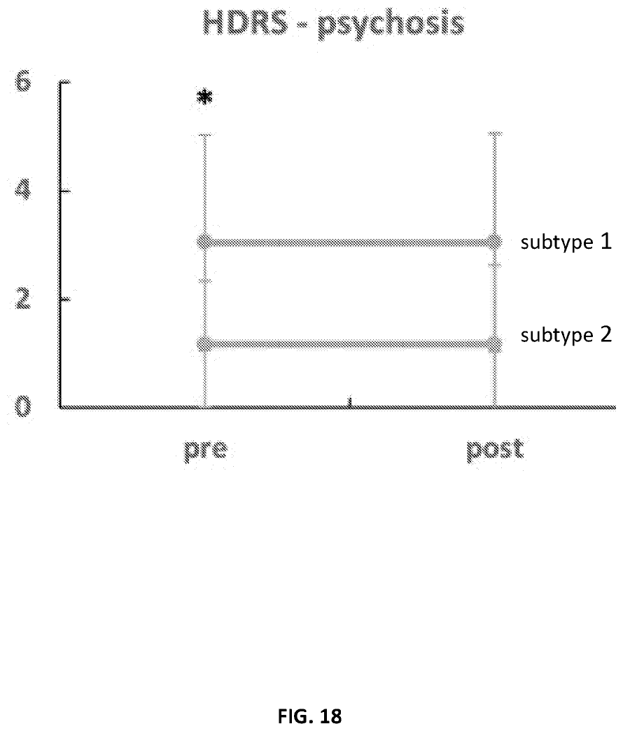

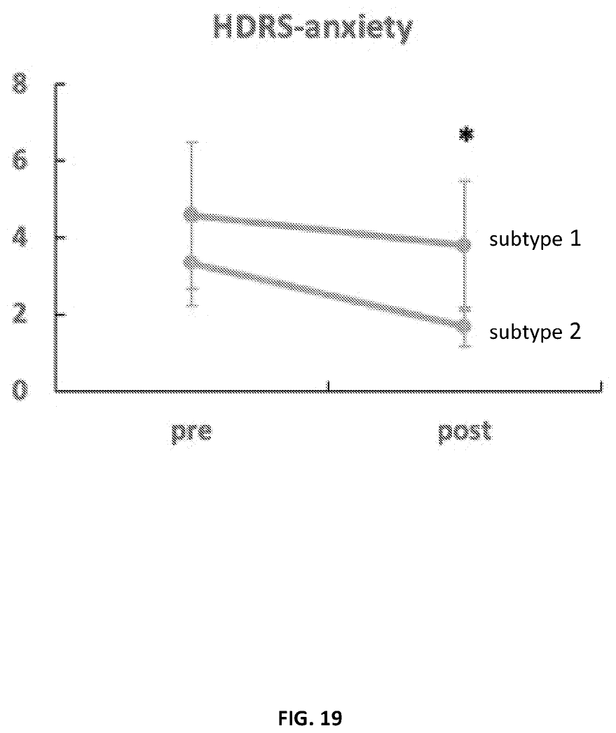

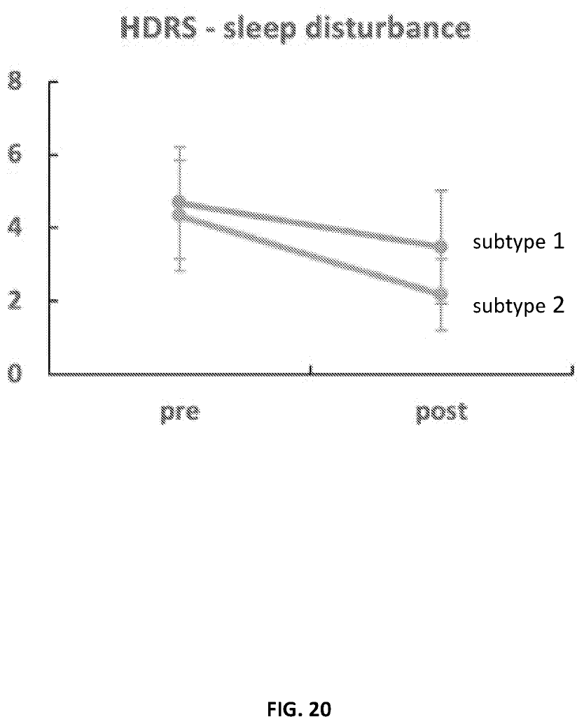

Before and after the TMS treatment, the Hamilton Depression Rating Scale (HDRS) of each subject was collected. Five factors (F1: Psychic depression; F2: Loss of motivated behavior; F3: Psychosis; F4: Anxiety; and F5: Sleep disturbance) were further calculated according to Milak et al.'s factor analysis results. (See Milak et al., “Neuroanatomic Correlates of Psychopathologic Components of Major Depressive Disorder” Archives of General Psychiatry 62, 397-408, (2005) for details). The total HDRS reduction, F3 (Psychosis), and F4 (Anxiety) of two subtypes are visualized in FIGS. 12-14 and exhibit a clear gradient. As shown in FIGS. 15-20, the total HDRS reduction and the change of the five factors pre- and post-TMS treatment are quantitatively compared between the two subtypes. Compared to subtype 1, subtype 2 has a significant reduction in HDRS total score, indicating a positive response to the TMS treatment. Subtype 1 shows a significantly higher value than subtype 2 in F3 (psychosis) factor (p<0.05, two-sample t-test) before TMS treatment, indicating a potential biomarker for TMS treatment outcome prediction. Besides, subtype 2 exhibits significant (p<0.05, two-sample t-test) relief of symptoms in F1 (psychiatric depression), F2 (loss of motivated behavior), and F5 (anxiety) after TMS treatment.

### Functional Connectivity Analysis

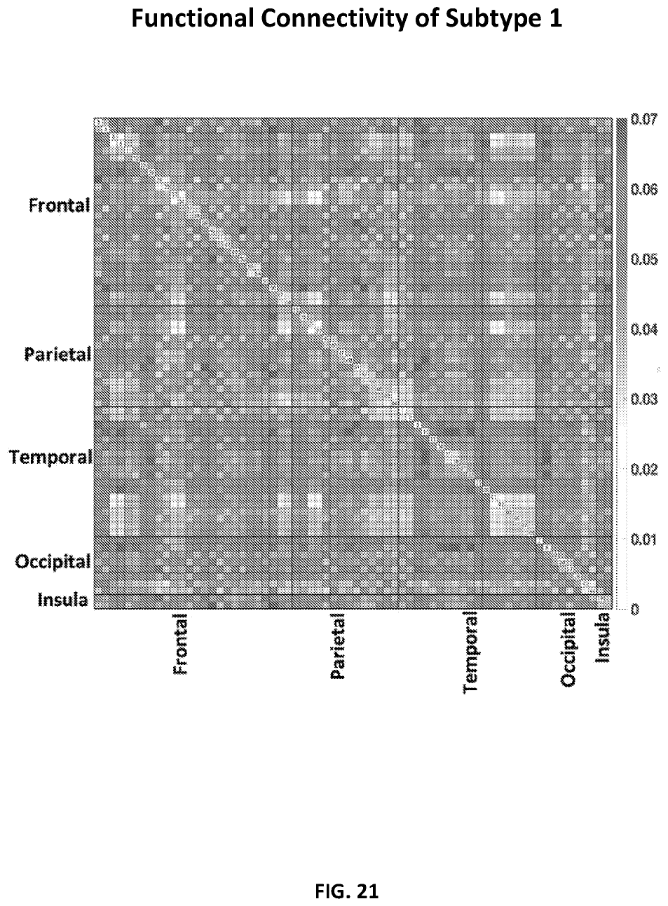

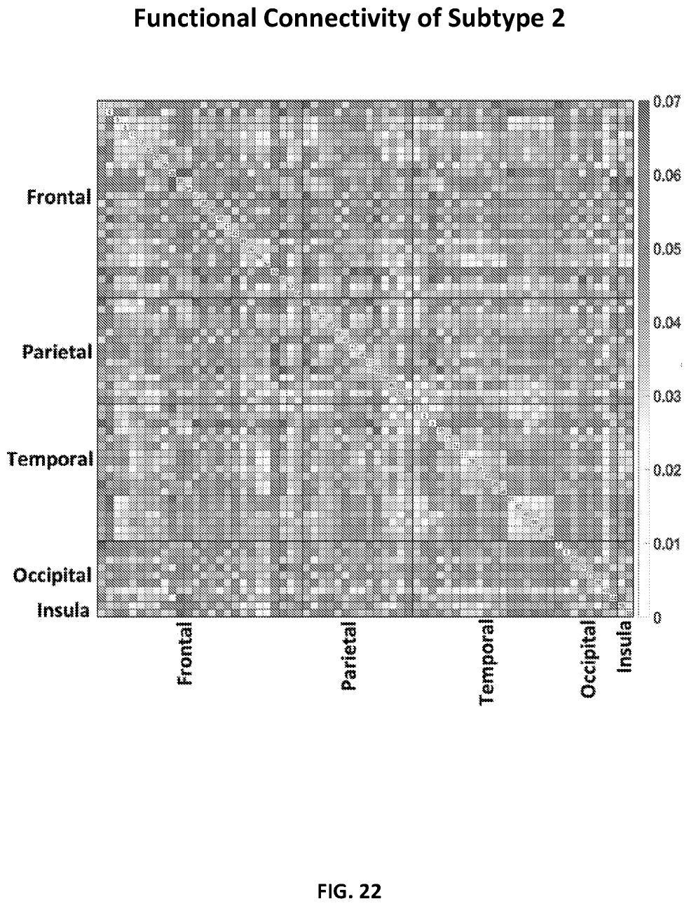

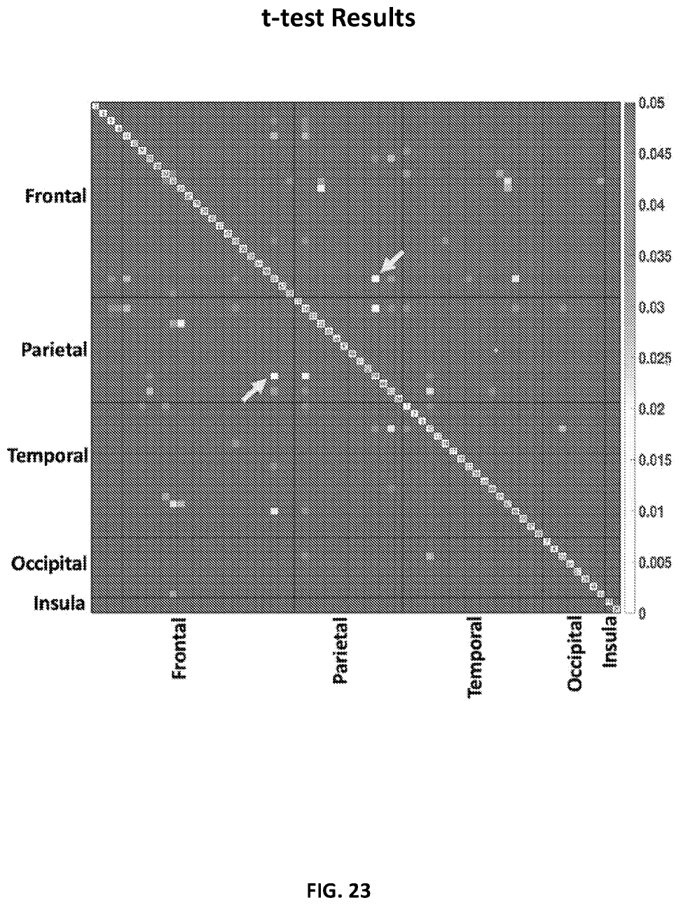

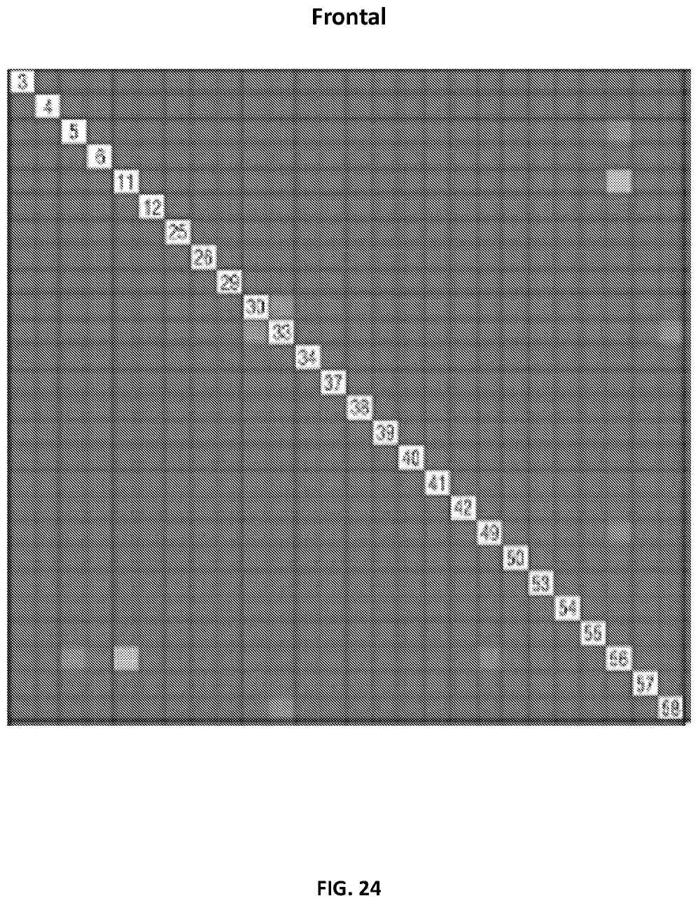

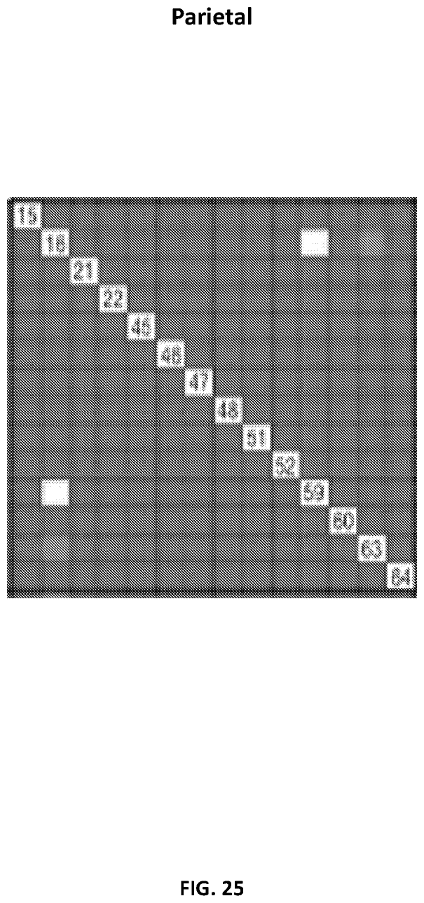

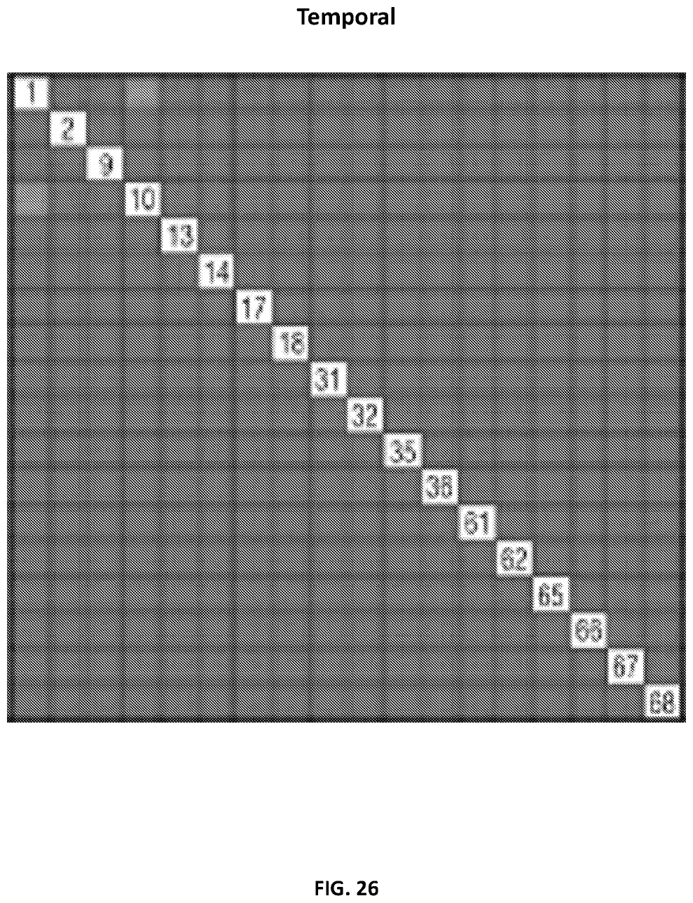

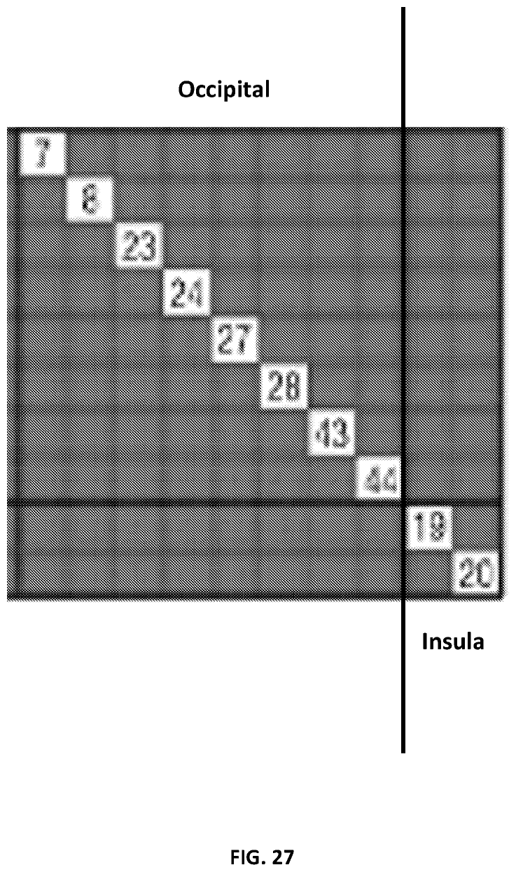

The coherence functional connectivity of the alpha-band (10 Hz) was calculated using the Fieldtrip toolbox. For source reconstruction, the precomputed MNI-standard Desikan-Killiany atlas was loaded (details shown in Table 3), a boundary element method (BEM) head model, and the source model. The whole-brain connectivity patterns based on the imaginary part of coherency are shown in FIGS. 21-27. The t-test statistic results show the connection between the right rostral middle frontal and left superior parietal shows the most significant difference in the two MDD subtypes (p<0.005, two-sample t-test). Functional connectivity of subtype 1 is shown in FIG. 21, subtype 2 is shown in FIG. 22 and t-test results are depicted in FIG. 23. The connection between ‘rostral middle frontal R’ (frontal) and ‘superior parietal L’ (parietal) shows significant differences between two subtypes (p<0.005, two-sample t-test) as are indicated by the arrows in the FIG. 23. FIGS. 24-27 show details of the diagonal of the matrix for the number region of interest based on the Desikan-Killiany atlas shown in Table 3. The diagonals for all three matrices show the same numbers, so the diagonal only for FIG. 23 is shown here, but the corresponding diagonals for FIGS. 21 and 22 are the same.

TABLE 3
Details of the Desikan-Killiany atlas

| # | Region of Interest (ROI) | Network |
|---|---|---|
| 1 | ‘bankssts L’ | Temporal |
| 2 | ‘bankssts R’ | Temporal |
| 3 | ‘caudalanteriorcingulate L’ | Frontal |
| 4 | ‘caudalanteriorcingulate R’ | Frontal |
| 5 | ‘caudalmiddlefrontal L’ | Frontal |
| 6 | ‘caudalmiddlefrontal R’ | Frontal |
| 7 | ‘cuneus L’ | Occipital |
| 8 | ‘cuneus R’ | Occipital |
| 9 | ‘entorhinal L’ | Temporal |
| 10 | ‘entorhinal R’ | Temporal |
| 11 | ‘frontalpole L’ | Frontal |
| 12 | ‘frontalpole R’ | Frontal |
| 13 | ‘fusiform L’ | Temporal |
| 14 | ‘fusiform R’ | Temporal |
| 15 | ‘inferiorparietal L’ | Parietal |
| 16 | ‘inferiorparietal R’ | Parietal |
| 17 | ‘inferiortemporal L’ | Temporal |
| 18 | ‘inferiortemporal R’ | Temporal |
| 19 | ‘insula L’ | Insula |
| 20 | ‘insula R’ | Insula |
| 21 | ‘isthmuscingulate L’ | Parietal |
| 22 | ‘isthmuscingulate R’ | Parietal |
| 23 | ‘lateraloccipital L’ | Occipital |
| 24 | ‘lateraloccipital R’ | Occipital |
| 25 | ‘lateralorbitofrontal L’ | Frontal |
| 26 | ‘lateralorbitofrontal R’ | Frontal |
| 27 | ‘lingual L’ | Occipital |
| 28 | ‘lingual R’ | Occipital |
| 29 | ‘medialorbitofrontal L’ | Frontal |
| 30 | ‘medialorbitofrontal R’ | Frontal |
| 31 | ‘middletemporal L’ | Temporal |
| 32 | ‘middletemporal R’ | Temporal |
| 33 | ‘paracentral L’ | Frontal |
| 34 | ‘paracentral R’ | Frontal |
| 35 | ‘parahippocampal L’ | Temporal |
| 36 | ‘parahippocampal R’ | Temporal |
| 37 | ‘parsopercularis L’ | Frontal |
| 38 | ‘parsopercularis R’ | Frontal |
| 39 | ‘parsorbitalis L’ | Frontal |
| 40 | ‘parsorbitalis R’ | Frontal |
| 41 | ‘parstriangularis L’ | Frontal |
| 42 | ‘parstriangularis R’ | Frontal |
| 43 | ‘pericalcarine L’ | Occipital |
| 44 | ‘pericalcarine R’ | Occipital |
| 45 | ‘postcentral L’ | Parietal |
| 46 | ‘postcentral R’ | Parietal |
| 47 | ‘posteriorcingulate L’ | Parietal |
| 48 | ‘posteriorcingulate R’ | Parietal |
| 49 | ‘precentral L’ | Frontal |
| 50 | ‘precentral R’ | Frontal |
| 51 | ‘precuneus L’ | Parietal |
| 52 | ‘precuneus R’ | Parietal |
| 53 | ‘rostralanteriorcingulate L’ | Frontal |
| 54 | ‘rostralanteriorcingulate R’ | Frontal |
| 55 | ‘rostralmiddlefrontal L’ | Frontal |
| 56 | ‘rostralmiddlefrontal R’ | Frontal |
| 57 | ‘superiorfrontal L’ | Frontal |
| 58 | ‘superiorfrontal R’ | Frontal |
| 59 | ‘superiorparietal L’ | Parietal |
| 60 | ‘superiorparietal R’ | Parietal |
| 61 | ‘superiortemporal L’ | Temporal |
| 62 | ‘superiortemporal R’ | Temporal |
| 63 | ‘supramarginal L’ | Parietal |
| 64 | ‘supramarginal R’ | Parietal |
| 65 | ‘temporalpole L’ | Temporal |
| 66 | ‘temporalpole R’ | Temporal |
| 67 | ‘transversetemporal L’ | Temporal |
| 68 | ‘transversetemporal R’ | Temporal |

### Event-Related Potential Analysis

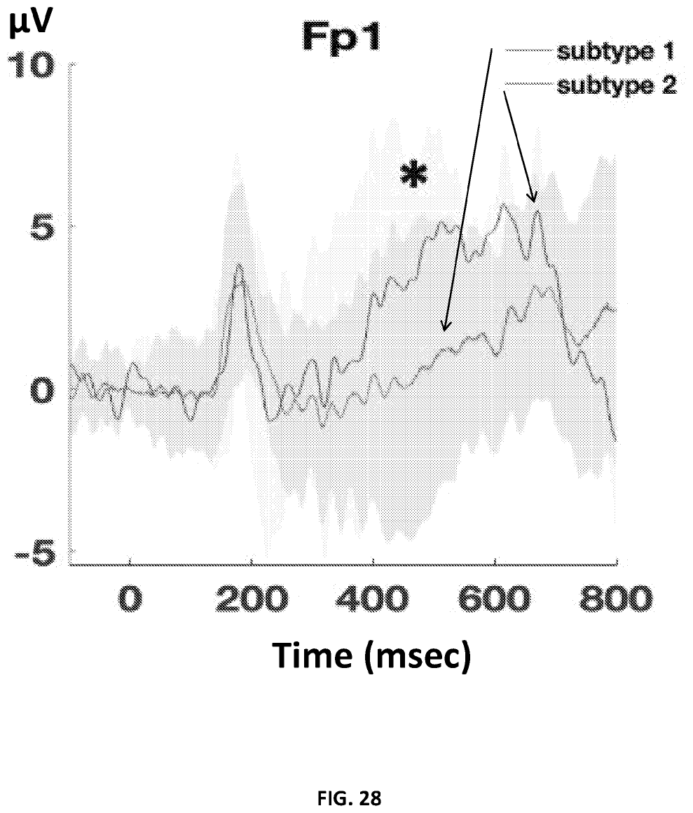

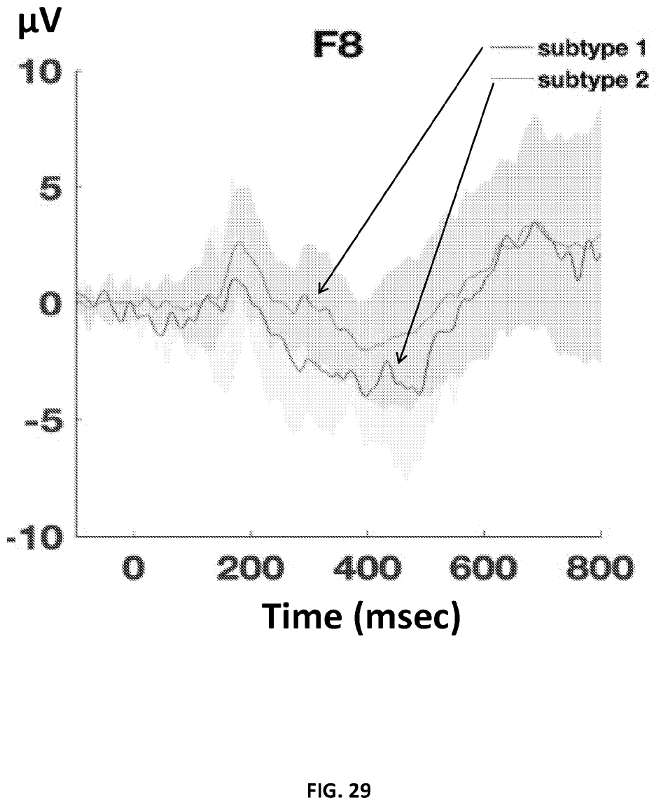

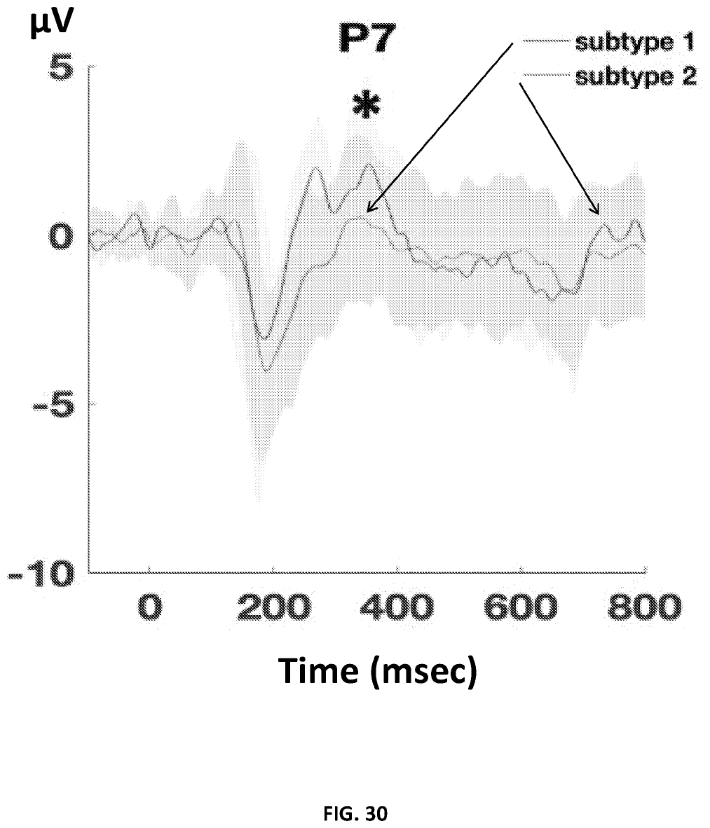

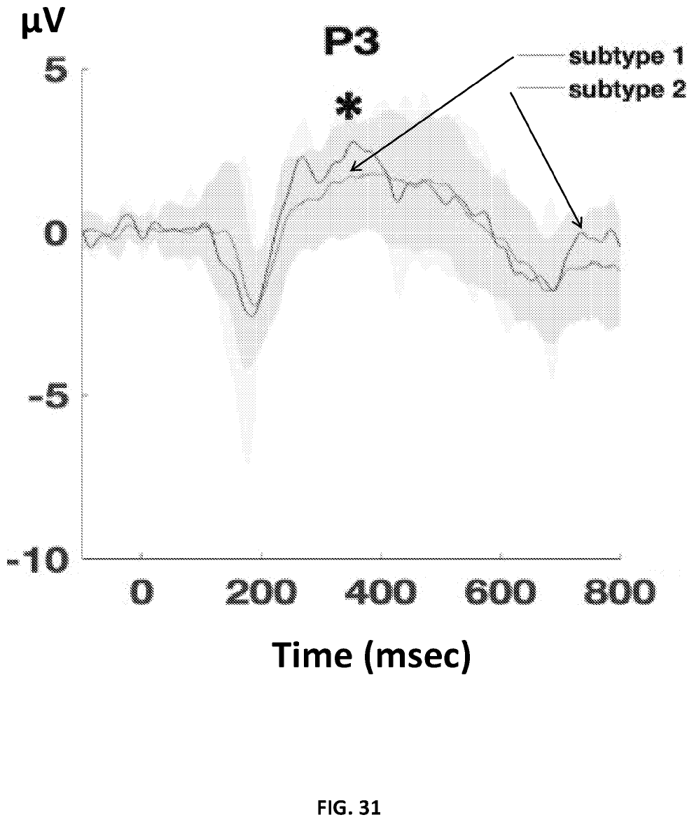

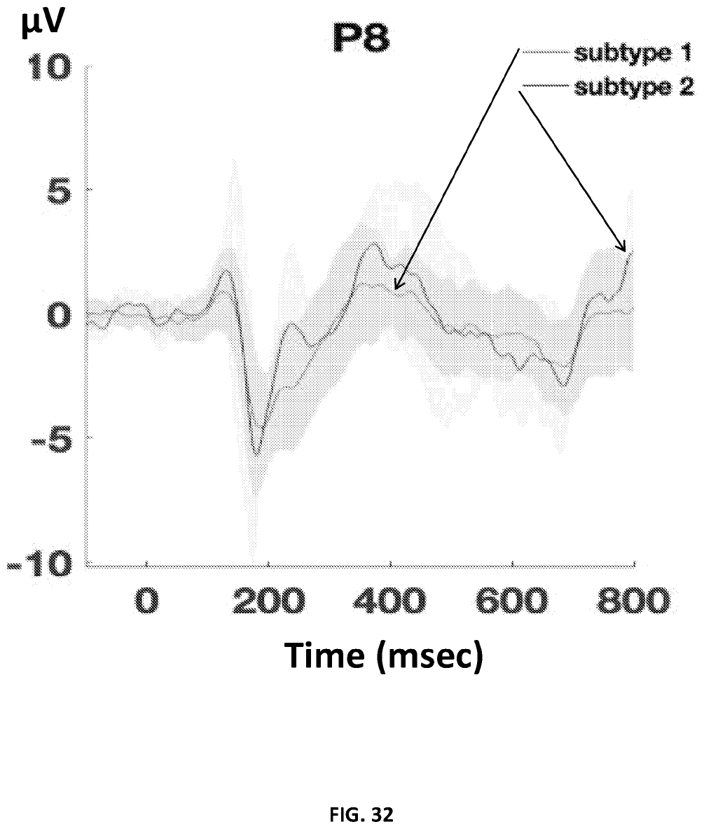

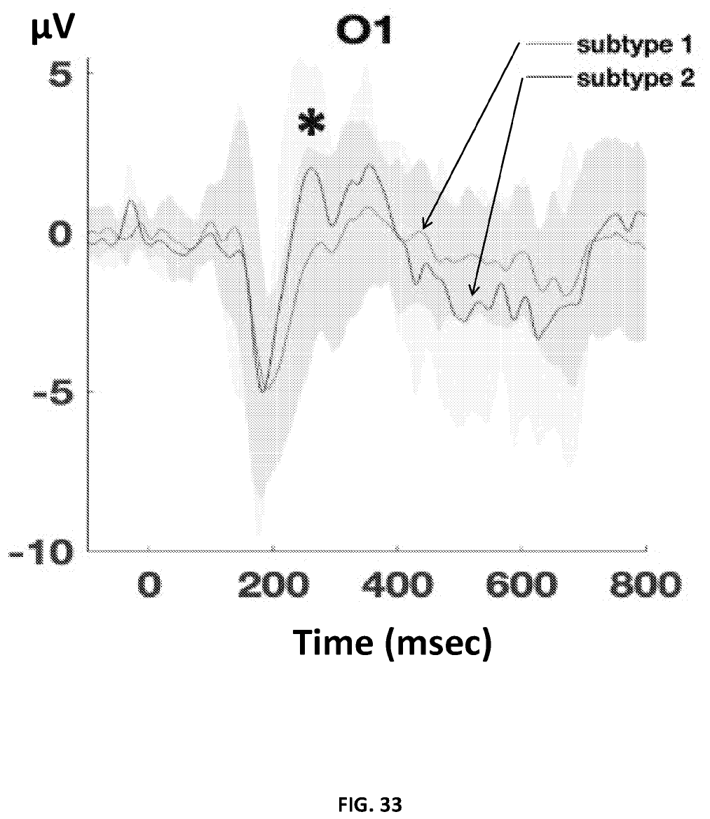

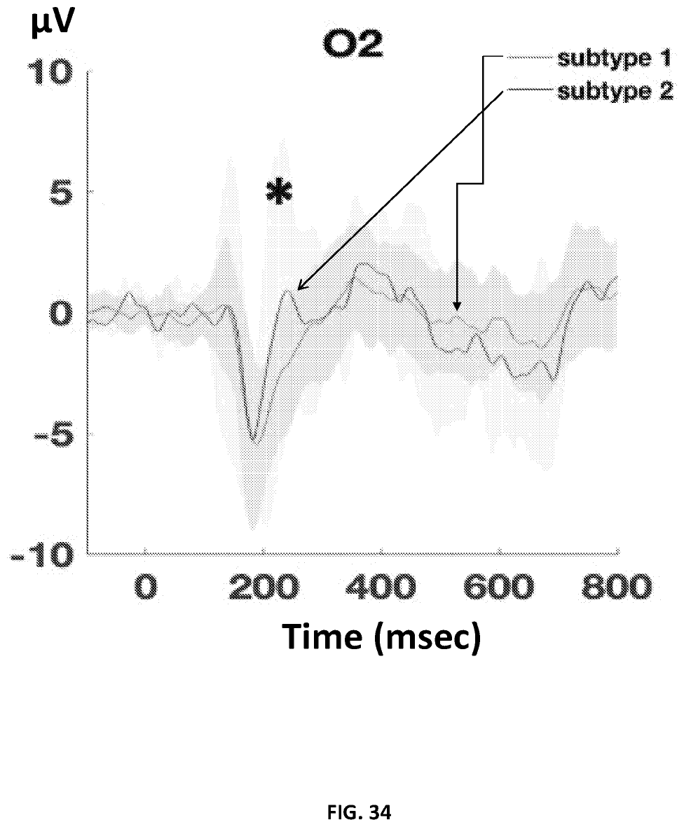

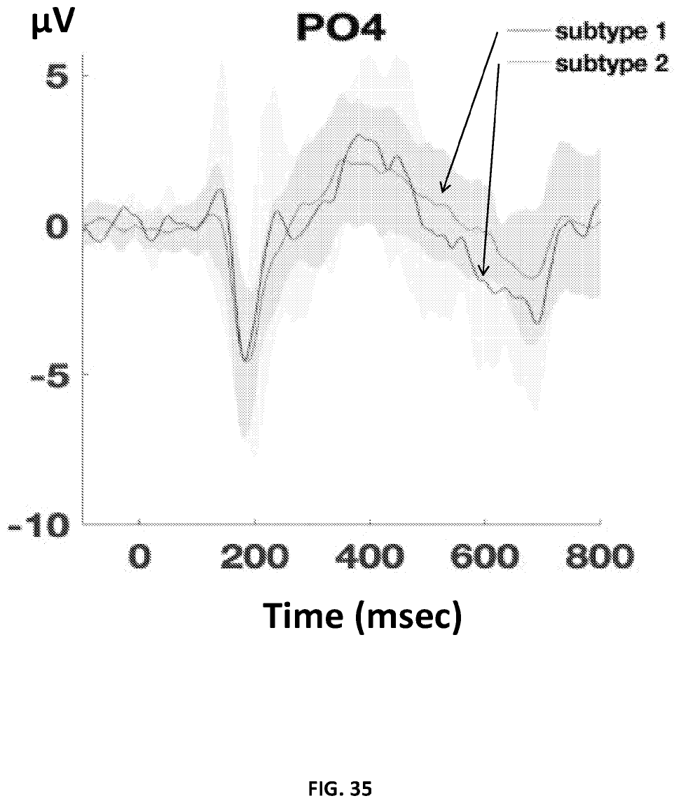

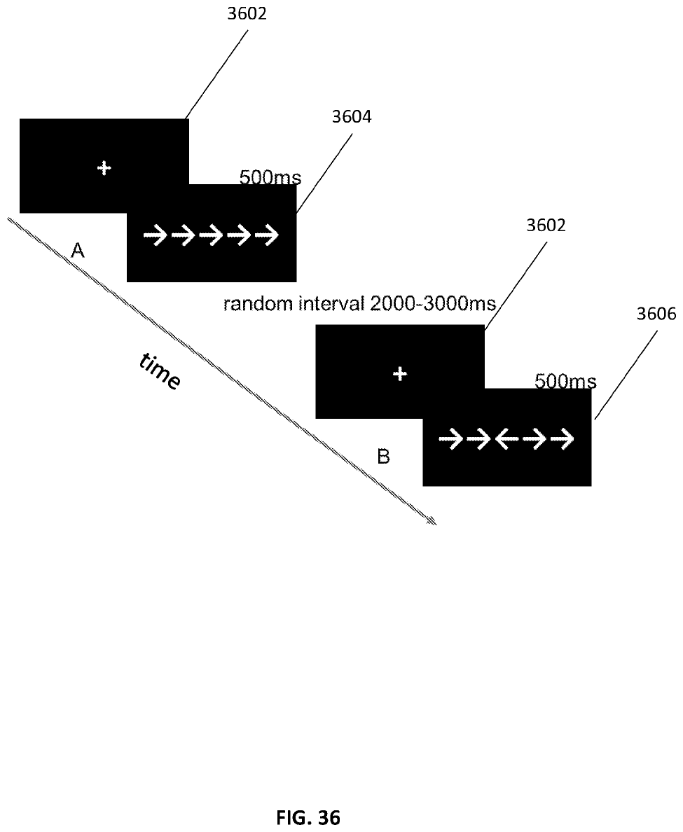

As shown in FIGS. 28-35, the subjects in two MDD subtypes behave differently in event related potentials (ERPs) when conducting arrow version flanker tasks. Some details of the flanker task are shown in FIG. 36 and more details supplied elsewhere in this disclosure. Compared to subtype 1, the subtype 2 patients show higher P300 amplitude on electrode Fp1 [F(1,23)=15.12; p=0.001], P3 [F(1,23)=5.806; p=0.026], P7 [F(1,23)=4.643; p=0.044], O1 [F(1,23)=5.669; p=0.027] and O2[F(1,23)=4.765; p=0.037], indicating that the subtype 2 had higher neural activity during the task.

### Participants and Data Acquisition

Multiple psychiatric disorders dataset (the first dataset). According to the Diagnostic and Statistical Manual of Mental Disorders (DSM-5), a total of 185 participants (all Chinese Han people) (BP=51, MDD=46, SZ=47, NC=41) were diagnosed after the semi-structured clinical interview by experienced psychiatrists (shown in Table 4). Patients in the first dataset 202 meet DSM-5 criteria for bipolar disorder, major depressive disorder, or schizophrenia. The participants were within the 14-40 age range, right-handed. Participants were excluded from enrollment if they had a currently active substance-use disorder, brain injury, a history of seizures, unstable medical condition, current pregnancy, prior electroconvulsive therapy. Healthy controls were recruited from the local community. The healthy controls also received semi-structured clinical interviews to exclude any current or lifetime evidence of psychiatric disorder. All participants in this study signed written informed consent, which was approved by the ethics committee of the institutional review board of Second Affiliated Hospital, Zhejiang University School of Medicine. Details of the protocols for EEG are shown in elsewhere in this disclosure.

TABLE 4
Demographic information of multiple psychiatric
disorders dataset (first dataset)

| | Bipolar | MDD | Schizophrenia | Controls |
|---|---|---|---|---|
| Gender (M/F) | 24/27 | 24/22 | 21/26 | 23/18 |
| Age (mean ± | 23.8 (7.7) | 23.0 (8.2) | 23.3 (6.5) | 24.8 (2.8) |
| (stdev)) | | | | |

TABLE 5
Demographic information of MDD TMS
treatment dataset (second dataset)

| | TMS response | TMS non-response |
|---|---|---|
| Gender (M/F) | 5/3 | 8/9 |
| Age (mean ± (stdev)) | 24.8 (6.7) | 25.5 (10/5) |

MDD with TMS treatment dataset (second dataset): All subjects (shown in Table 5) were diagnosed as MDD by experienced psychiatrists after a semi-structured clinical interview and DSM-5. The subjects were within the 16-48 age range, right-handed who were screened for ethical clearance, with only Chinese Han people. Specific clinical symptoms were evaluated using the Hamilton depression rating scale. The EEG signals were recorded using the same equipment as the first dataset 202. In addition, all the MDD patients participated the event-related potential (ERP) task (the Arrow version flanker task, more details of the task can be found below). Hamilton depression rating scales were rated before and after a complete course of TMS treatment. The TMS protocol involved 5 consecutive days of two intermittent theta-burst stimulation (iTBS) sessions per day (1,800 pulses per session, the interval between each session is above 4 hours) delivered to the region of the left dorsolateral prefrontal cortex (left DLPFC). The iTBS was performed using a MagPro X100 stimulator (Mag Venture, Georgia, USA) with a figure-of-eight coil, 70 mm diameter. The stimulus intensity is 110%˜120% of the resting motor threshold.

Details of the flanker task. FIG. 36 shows some details of the flanker task. In each trial, participants viewed five white arrows presented for 500 ms against a black background as shown in FIG. 36. The participants were asked to respond as quickly and as accurately as possible to indicate the direction of the middle arrow by pressing the left or right mouse button. Participants had up to 3000 ms from the onset of the stimulus to respond. 200 of the trials were congruent 3604 in that all the arrows were pointing in the same direction (←←←←← or →→→→→) (as in part A of FIG. 36), whereas the other 40 trials were incongruent 3606 (e.g., ←←→←← or →→←→→) (as in Part B of FIG. 36). The trial order was random for each participant and the inter-trial interval varied from 2000 to 3000 ms, during which time participants viewed a white fixation cross 3604 centered on a black background. Participants received short breaks every 80 trials throughout the task.

### Major Depressive Disorder Subtype Discovery

By training the MCRNN on the first dataset 202 in a supervised way, the model has learned the way to nonlinearly map the EEG features from its original feature space to a subspace in which the differences among psychiatric disorders are more distinctive. To make full use of the advantage of MCRNN, the trained MCRNN was applied to an independent MDD dataset for subtype discovery. The procedures are as follows: After optimizing the MCRNN model using The first dataset 202, the model parameters of the MCRNN were saved. The EEG signals of each subject in The second dataset 204 were first preprocessed using the same procedure as The first dataset 202, then the epochs were sent to the trained MCRNN model for extracting the hidden state feature. As a result, for each EEG epoch in The second dataset 204, a vector representation consisting of 32 elements was obtained. The epochs were pooled together for t-SNE clustering and subtype discovery.

### Discussion

Due to the intrinsic heterogeneity and strong comorbidities in neurobiological abnormalities within current clinical definitions for diagnosis, discovering objective psychiatric biomarkers from neuroimaging is vital for understanding pathophysiology and improving treatment. In this disclosure, a new deep learning-based model, MCRNN, for identifying multiple psychiatric disorders using rsEEG is described. An average accuracy of 52.4% was achieved in the 4-class classification task, which is significantly above that expected by chance alone (25%). The spectrum continuum of psychiatric disorders was then visualized using t-SNE based on the extracted rsEEG features. Subsequently, the MCRNN optimized on the multiple psychiatric disorder dataset was applied to an independent MDD dataset, leading to the discovery of an MDD subtype which is sensitive to the TMS treatment. The various subtypes also exhibited significant differences in Hamilton depression rating scores, functional connectivity patterns, and neural plasticity.

It is challenging to quantify diagnosis based on the clinical symptoms. The current classification diagnostic criteria (ICD and DSM) are based on the evaluation of clinical symptoms. The same group of similar symptoms may be caused by completely different biological processes. The present disclosure classified mental diseases based on objective neurophysiological markers. As a neural activity recording tool with high time resolution, rsEEG can classify the neural activities of different mental diseases at the systematic level. Combined with the state-of-the-art deep learning methods, EEG exhibits great prospects in facilitating the accuracy of clinical diagnosis by revealing the mechanisms of psychiatric disorders. Compared to conventional time-frequency-based analysis which manually extracts different frequency bands of rsEEG to generate functional connectivity features, the convolutional module of the MCRNN can automatically learn spatial filters to map the original EEG into subspaces. The recurrent module subsequently integrates the sequential information for accurate classification. The 4-class results demonstrate that the MCRNN can efficiently capture the discriminative neural activity patterns among psychiatric mental disorders (e.g., MDD, BP, SZ). By analyzing the 4-class confusion matrix and the psychiatric disorder visualization, it was found that BP and MDD have more bilateral overlaps than others. This phenomenon occurs because BP and MDD have many overlaps in clinical symptoms, and the core symptom of MDD can also be found in the depressive or mixed states of BP. With this deep learning model, the specific stages of the disease (such as prodromal stage, initial stage, and chronic disease stage) can be quantified, visualized, and compared. In addition, the treatment response can be accurately predicted, which benefits patients with accurate individualized intervention. In this example, the DSM label was used as the ground truth to train the 4-class deep learning model, this procedure introduced some biases to the model and would be overcome using an unsupervised learning approach in the future.

The ideal biomarkers should be both sensitive and specific for identifying mental disorders. By interpreting the deep learning model, the bilateral frontal (Fp1, F8) and parietal-occipital (P7, P3, P8, PO4, O1, O2) channels activity were found to contribute most to the classification task. The revealed channels which contributed most to the 4-class classification task coincide with other resting and task-related EEG studies. For example, by studying the averaged power spectra between schizophrenia patients, Etevenon et al. reported alpha peak and the mean RMS amplitude is higher over P3-01 than over P4-02 for the residual-type of schizophrenic patients, when compared to his matched control sub-group of high-alpha subjects which presented almost symmetrical occipital alpha peaks and RMS amplitudes (see Etevenon, P. “Intra and inter-hemispheric changes in alpha intensities in EEGs of schizophrenic patients versus matched controls”. Biological Psychology 19, 247-256, (1984).). Other researchers found that the mean frequency is higher at Fp1 and Fp2 in bipolar disorder patients than in the control group, but no differences were found in frequencies between schizophrenic or schizoaffective patients against the control group (Wix-Ramos, R. et al. “Drug Treated Schizophrenia, Schizoaffective and Bipolar Disorder Patients Evaluated by qEEG Absolute Spectral Power and Mean Frequency Analysis”. Clin Psychopharmacol Neurosci 12, 48-53 (2014)). In addition, the P300 measurement, a physical marker of neural growth factor level related to neural plasticity obtained from event-related potential, has the potential to be a biomarker for mental disorder classification. In the present disclosure, MDD subtype 2 displayed a significantly higher P300 peak than subtype 1 in the selected channels, indicating that neural plasticity may highly correlate with TMS treatment outcome.

The discovery of psychiatric subtypes is challenging because the discriminative features are easily merged by confounds (e.g., age, gender, site effects). The key to a successful subtype discovery is to appropriately map the features from the source space to a subspace in which the heterogeneity of disorders can be maximized. Given the circumstances, deep learning is an ideal solution for two reasons: First, deep learning architecture is quite flexible and suitable for various feature dimensions. Second, due to the gradient-descent and error back-propagation optimizing strategy, deep learning algorithms can automatically learn the manifold from the training data, which has been proved the right projection to the disease-related subspace. In the present disclosure, the two discovered MDD subtypes demonstrate the effectiveness of the proposed MCRNN in learning high-level psychiatric-related features, and the two identified subtypes show significant differences in TMS treatment outcomes. The rsEEG features extracted using the MCRNN before treatment accurately predicted the TMS outcome (ACC=84%), validating the prediction and demonstrating the model's great potential in reducing medical costs and patients' suffering. By comparing the HDRS of two discovered subtypes, it was found that patients with lower psychosis factors before TMS treatment are more likely to experience relief after TMS treatment. Therefore, the psychosis factor can also be used as a predictor because it shows significant differences before TMS treatment, coinciding with previous findings that MDD patients with higher anxiety or psychotic features are more likely to result in being diagnosed with TMS treatment-resistant depression. MDD is characterized by hypoconnectivity within the frontoparietal network (FN), a set of regions involved in cognitive control of attention and emotion regulation, and hypoconnectivity between frontoparietal systems and parietal regions of the dorsal attention network (DAN) involved in attending to the external environment. The MDD subtype discovery results also revealed the abnormal connection between right rostral middle frontal and left superior parietal. Besides, by comparing the event-related potential of two subtypes, it was found that subtype 2 exhibits significantly stronger neural plasticity than subtype 1 during the attention task. This may also explain the better TMS treatment outcomes of subtype 2.

There exist several limitations and potential extensions of the present disclosure. Even though the ideal way of discovering the MDD subtypes is by applying an unsupervised clustering method to the original rsEEG features, clustering based on the original EEG signals is very likely to be misled by confounds due to the low signal-to-noise ratio of EEG. The application of the MCRNN is a compromise way which takes both feature mapping and unsupervised clustering together into account. Besides, with more MDD subjects collected in some other data sites/hospitals, the subtype results can be further validated. In addition, multi-modal fusion would provide more complementary information which can further validate the EEG finding. For future work, given the flexibility of the MCRNN in processing temporal data, it can be further applied to classify magnetoencephalogram (MEG) data.

In summary, the described MCRNN provides an affordable plan for identifying psychiatric disorders based on resting-state EEG. Combining with the visualization techniques, the intrinsic relationships among psychiatric disorders are revealed, which is helpful for further understanding their comorbidities. In addition, by leveraging the spatiotemporal information of the rsEEG and inhibiting the disease-irrelevant confounds, the MDD subtype which is sensitive to TMS treatment is separated from the whole group, showing great promise in promoting the understanding of major psychiatric disorders and facilitating the personalized interventions for individuals with psychiatric disorders.

### Details of the EEG Protocols

The EEG signal was recorded in a quiet room with semi-darkened light, moderated temperature and humidity, good ventilation, and electromagnetic shielding. Subjects were asked to sit on a chair in their own comfortable way, with eyes closed while staying awake. EEG data were recorded using a wired Waveguard cap containing 64 Ag/AgCl recording channels (ANT Neuro, Hengelo, Netherlands), shown in Table 6, below. EEG recording electrodes were located at standard locations following the 10/20 international placement system. Signals were sampled at 1 kHz, referenced relative to CPz, online grounded at AFz, and amplified with an Eego™ amplifier (ANT Neuro, Hengelo, Netherlands). Before each recording, scalp impedances were monitored to ensure impedance levels were below 20 kΩ. The electrodes placed at the supra-orbital to the left eye were the bipolar recordings of electro-ocular activity (EOG). Resting-state EEG was continuously recorded for 5 minutes.

TABLE 6
Details of the EEG electrodes and network

| Electrode | Network | Electrode | Network | Electrode | Network | Electrode | Network |
|---|---|---|---|---|---|---|---|
| ‘Fp1’ | Frontal | ‘C4’ | Central | ‘AF3’ | Frontal | ‘P5’ | Parietal |
| ‘Fpz’ | None | ‘T8’ | Temporal | ‘AF4’ | Frontal | ‘P1’ | Parietal |
| ‘Fp2’ | Frontal | ‘M2’ | Nonc | ‘AF8’ | Frontal | ‘P2’ | Parictal |
| ‘F7’ | Frontal | ‘CP5’ | Central | ‘F5’ | Frontal | ‘P6’ | Parietal |
| ‘F3’ | Frontal | ‘CP1’ | Central | ‘F1’ | Frontal | ‘PO5’ | Occipital |
| ‘Fz’ | None | ‘CP2’ | Central | ‘F2’ | Frontal | ‘PO3’ | Occipital |
| ‘F4’ | Frontal | ‘CP6’ | Central | ‘F6’ | Frontal | ‘PO4’ | Occipital |
| ‘F8’ | Frontal | ‘P7’ | Parietal | ‘FC3’ | Central | ‘PO6’ | Occipital |
| ‘FC5’ | Central | ‘P3’ | Parietal | ‘FCz’ | None | ‘FT7’ | Temporal |
| ‘FC1’ | Central | ‘Pz’ | None | ‘FC4’ | Central | ‘FT8’ | Temporal |
| ‘FC2’ | Central | ‘P4’ | Parietal | ‘C5’ | Central | ‘TP7’ | Temporal |
| ‘FC6’ | Central | ‘P8’ | Parietal | ‘C1’ | Central | ‘TP8’ | Temporal |
| ‘M1’ | None | ‘POz’ | None | ‘C2’ | Central | ‘PO7’ | Occipital |
| ‘T7’ | Temporal | ‘O1’ | Occipital | ‘C6’ | Central | ‘PO8’ | Occipital |
| ‘C3’ | Central | ‘O2’ | Occipital | ‘CP3’ | Central | ‘Oz’ | None |
| ‘Cz’ | None | ‘AF7’ | Frontal | ‘CP4’ | Central | | |

### Details of EEG Preprocessing

Resting-state EEG: In an example, EEGLAB was used for EEG processing, although other analysis software may also be employed. The procedures were as follows: 1) the first 5 seconds of EEG, which were not stable, are discarded; 2) a bandpass filter with a low cut-off of 0.5 Hz and high cut-off of 70 Hz is adopted for eliminating low-frequency drift and high-frequency noise; 3) the power supply AC noise is removed (e.g., 60 Hz in the United States, 50 Hz in China, etc.); 4) down-sample to 250 Hz; 5) convert the EEG dataset to average reference; 6) remove or correct any remaining artifacts including flatline channels, low-frequency drifts, noisy channels, short-time bursts; and 7) the EEG data are then separated into 1-sec epochs. After these preprocessing procedures, 260˜300 epochs per subject are obtained for further analysis. Each epoch is represented with a 64×250 matrix. The rsEEG signals in The first dataset 202 and The second dataset 204 were preprocessed using the same pipeline.

Event-related potential (ERP) analysis: In an example, EEGLAB was also used for processing the ERP data. The exemplary procedures were as follows: 1) the first 5-seconds of EEG which is not stable are discarded; 2) a bandpass filter with a low cut-off of 0.5 Hz and a high cut-off of 70 Hz is adopted to eliminate low-frequency drift and high-frequency noise; 3) the power supply AC noise is removed (e.g., 60 Hz in the United States, 50 Hz in China, etc.); 4) independent component analysis is run and the artifacts of eye movement and muscle are removed; and 5) the epochs are extracted. Each epoch contains samplings from 100 ms before to 800 ms after each trigger. In this example, a total of 240 epochs were extracted for further analysis (incongruent trails=40, congruent trails=200).

### Dimensionality Reduction & Data Visualization

The visualization of MCRNN was performed by the unsupervised clustering or dimensionality reduction technique t-SNE, which embeds high-dimensional data into a low-dimensional space while preserving the pairwise distances of the data points, implemented on the MATLAB platform. The output of the last hidden layer of the MCRNN was used for t-SNE clustering. The parameters for the stochastic optimization for t-SNE are as follows: When visualizing the spectrum of all 4-class samples, the perplexity is set to 250. When the t-SNE was used for MDD subtype discovery, the perplexity is set to 200. Other parameters were as default. The distance metric was the Euclidian distance.

Many operating systems, including Linux, UNIX®, OS/2®, and Windows®, are capable of running many tasks at the same time and are called multitasking operating systems. Multi-tasking is the ability of an operating system to execute more than one executable at the same time. Each executable is running in its own address space, meaning that the executables have no way to share any of their memory. Thus, it is impossible for any program to damage the execution of any of the other programs running on the system. However, the programs have no way to exchange any information except through the operating system (or by reading files stored on the file system).

Multi-process computing is similar to multi-tasking computing, as the terms task and process are often used interchangeably, although some operating systems make a distinction between the two. The present invention may be a system, a method, and/or a computer program product at any possible technical detail level of integration. The computer program product may include a computer readable storage medium (or media) having computer readable program instructions thereon for causing a processor to carry out aspects of the present invention. The computer readable storage medium can be a tangible device that can retain and store instructions for use by an instruction execution device.

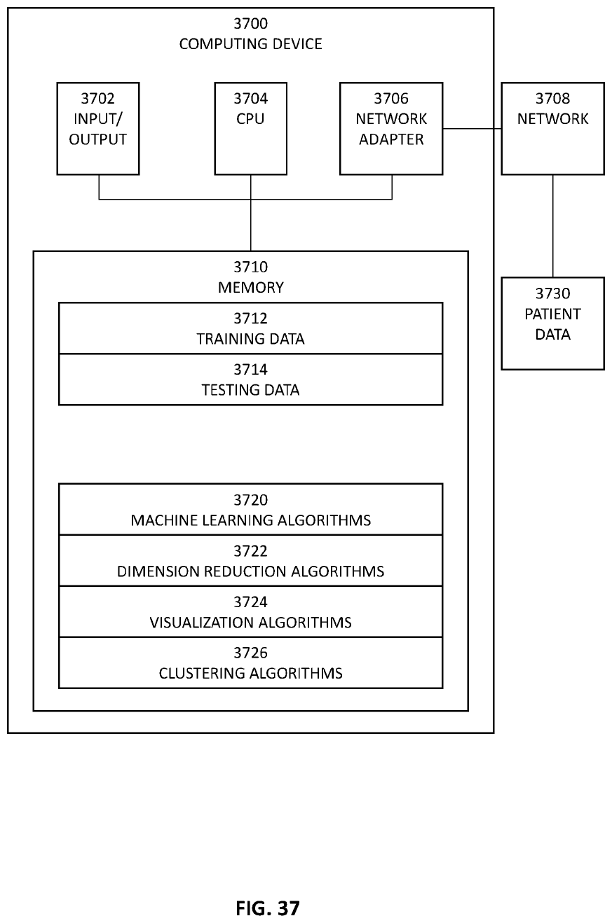

An exemplary computing device is illustrated in FIG. 37. The computing device 3700 may comprise an input/output portion 3702, a central processing unit 3704, although other processors may also be used, such as graphics processing units, or multiple processors, to facilitate the calculations involved with this system or method. A network adapter 3706 enables the computing device to communicate with other devices on a network 3708. This network connection may comprise a wired connection, a connection using fiber optical components, or may be a wireless connection. The patient data 3730 may come from a database or may come from a measurement tool such as an EEG cap. Within the computer memory 3710, there may be various types of data such as training data 3712 used to train a machine learning algorithm 3720. The computer memory 3710 may also have testing data 3714, which is used to test or validate a machine learning algorithm 3720. In addition, there may be algorithms for dimension reduction 3722, for visualization 3724, or for clustering 3726.

The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing.

A computer readable storage medium, as used herein, is not to be construed as being transitory signals per se, such as radio waves or other freely propagating electromagnetic waves, electromagnetic waves propagating through a waveguide or other transmission media (for example, light pulses passing through a fiber-optic cable), or electrical signals transmitted through a wire. Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers, and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions. These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks. The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks. The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s).

In some alternative implementations, the functions noted in the blocks may occur out of the order noted in the Figures. For example, two blocks shown in succession may, in fact, be executed substantially concurrently, or in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or that carry out combinations of special purpose hardware and computer instructions. Although specific embodiments of the present invention have been described, it will be understood by those of skill in the art that there are other embodiments that are equivalent to the described embodiments. Accordingly, it is to be understood that the invention is not to be limited by the specific illustrated embodiments, but only by the scope of the appended claims.

From the above description, it can be seen that the present invention provides a system, computer program product, and method for the efficient execution of the described techniques. References in the claims to an element in the singular is not intended to mean “one and only” unless explicitly so stated, but rather “one or more.” All structural and functional equivalents to the elements of the above-described exemplary embodiment that are currently known or later come to be known to those of ordinary skill in the art are intended to be encompassed by the present claims.

While the foregoing written description of the invention enables one of ordinary skill to make and use what is considered presently to be the best mode thereof, those of ordinary skill will understand and appreciate the existence of alternatives, adaptations, variations, combinations, and equivalents of the specific embodiment, method, and examples herein. Those skilled in the art will appreciate that the within disclosures are exemplary only and that various modifications may be made within the scope of the present invention. In addition, while a particular feature of the teachings may have been disclosed with respect to only one of several implementations, such feature may be combined with one or more other features of the other implementations as may be desired and advantageous for any given or particular function. Furthermore, to the extent that the terms “including”, “includes”, “having”, “has”, “with”, or variants thereof are used in either the detailed description and the claims, such terms are intended to be inclusive in a manner similar to the term “comprising.”

Other embodiments of the teachings will be apparent to those skilled in the art from consideration of the specification and practice of the teachings disclosed herein. The invention should therefore not be limited by the described embodiment, method, and examples, but by all embodiments and methods within the scope and spirit of the invention. Accordingly, the present invention is not limited to the specific embodiments as illustrated herein, but is only limited by the following claims.

What is claimed is:

1. A method of diagnosing a mental illness in a patient comprising:
measuring a plurality of encephalography signals from the patient;
feeding the plurality of encephalography signals into a trained multi-scale recurrent neural network;
classifying, by the trained multi-scale recurrent neural network, the patient as having a selected mental illness of a plurality of mental illnesses; and
communicating the selected mental illness to a user.

2. The method of claim 1, wherein the plurality of encephalography signals comprises at least one electroencephalography (EEG) signal or at least one magnetoencephalography (MEG) signal.

3. The method of claim 2, wherein the at least one EEG signal comprises at least one of the bilateral frontal (Fp1, F8) channels or the parietal-occipital (P7, P3, P8, PO4, O1, O2) channels.

4. The method of claim 1, wherein the plurality of mental illnesses comprises at least major depressive disorder, bipolar disorder, schizophrenia, and normal control.

5. The method of claim 1, wherein a feature is extracted from the multi-scale recurrent neural network and the feature is applied to a second machine learning model to classify the feature as a sub-type of the selected mental illness.

6. The method of claim 1, further comprising incorporating other patient information as part of an input into the multi-scale recurrent neural network.

7. The method of claim 1, further comprising, if the patient is classified as having major depressive disorder:
reducing the dimensionality of the plurality of encephalography signals;
visualizing the dimensionality-reduced plurality of encephalography signals;
classifying the patient as belonging to a sub-type of major depressive disorder based on the visualization; and
communicating the sub-type to the user.

8. The method of claim 7, wherein the sub-type comprises a major depressive disorder treatable with transcranial magnetic stimulation.

9. The method of claim 7, wherein the method of dimensionality reduction and the method for visualization comprise t-distributed stochastic neighbor embedding (t-SNE).

10. A system comprising:
a computer having a processor and memory;
a user device;
a measurement device;
the computer, the user device and the measurement device configured together to perform a method comprising:
measuring, at the measurement device, a plurality of encephalography signals from a patient;
feeding, at the computer, the plurality of encephalography signals into a trained multi-scale recurrent neural network;
classifying, by the trained multi-scale recurrent neural network, the patient has having a selected mental illness of a plurality of mental illnesses; and
communicating the selected mental illness to the user device.

11. The system of claim 10, wherein the plurality of encephalography signals comprises at least one electroencephalography signal or at least one magnetoencephalography signal.

12. The system of claim 11, wherein the at least one EEG signal comprises at least one of the bilateral frontal (Fp1, F8) channels or the parietal-occipital (P7, P3, P8, PO4, O1, O2) channels.

13. The system of claim 10, wherein the plurality of mental illnesses comprises at least major depressive disorder, bipolar disorder, schizophrenia, and normal control.

14. The system of claim 10, wherein the computer extracts a feature from multi-scale recurrent neural network and applies the feature to a second machine learning model to classify a sub-type of the selected mental illness.

15. The system of claim 10, wherein the computer further incorporates other patient information as part of the input into the multi-scale recurrent neural network.

16. The system of claim 10, wherein, if the patient is classified as having major depressive disorder, the computer further performs the steps of:
reducing the dimensionality of the plurality of encephalography signals;
visualizing the dimensionality-reduced plurality of encephalography signals;
classifying the patient as belonging to a sub-type of major depressive disorder based on the visualization; and
communicating the sub-type to the user.

17. The system of claim 16, wherein the sub-type comprises a major depressive disorder treatable with transcranial magnetic stimulation.

18. The system of claim 16, wherein the dimensionality reduction and visualization comprises t-distributed stochastic neighbor embedding (t-SNE).
